(19) United States
(12) Patent Application Publication
(10) Pub. No.: US 2021/0154208 A1
(43) Pub. Date: May 27, 2021

(54) Method to Identify Agents That Can Overcome Inhibition Caused By Drug-protein Binding of the Human Plasma Protein, Alpha-1-acid Glycoprotein

(71) Applicant: The Johns Hopkins University, Baltimore, MD (US)

(72) Inventors: David Joseph Young, Baltimore, MD (US); Donald Small, Pikesville, MD (US); Jun O. Liu, Clarksville, MD (US)

(21) Appl. No.: 17/100,721

(22) Filed: Nov. 20, 2020

Related U.S. Application Data
(60) Provisional application No. 62/940,520, filed on Nov. 26, 2019.

(51) Int. Cl. A61K 31/57; A61K 31/407

(52) U.S. Cl. CPC A61K 31/57; A61K 31/407

(57) ABSTRACT

Described are methods of inhibiting one or more pharmaceutical agent from binding to a plasma protein so it binds to its target. Specifically, a plasma protein binding agent is administered prior to, concurrently with, or after the administration of one or more pharmaceutical agents that binds to a plasma protein in vivo. The methods of the present invention allow pharmaceutical agents that bind to plasma binding proteins to treat disease cells in vivo.

### CROSS-REFERENCE TO RELATED APPLICATIONS

This application claims the benefit under 35 USC § 119(e) to U.S. Provisional Application Ser. No. 62/940,520 filed Nov. 26, 2019. The disclosure of the prior application is considered part of and is incorporated by reference in the disclosure of this application.

### STATEMENT OF GOVERNMENTAL INTEREST

This invention was made with government support under grant nos. CA060441 and CA090668 awarded by the National Institutes of Health. The government has certain rights in the invention.

### BACKGROUND OF THE INVENTION

The FMS-like tyrosine kinase 3 (FLT3) is the most frequently mutated gene in acute myeloid leukemia (AML), with approximately one third of adult cases of AML and a lower fraction of pediatric AML cases expressing a FLT3 mutation. Wild-type FLT3 is expressed within early progenitor populations and may also be expressed at a lower level within the hematopoietic stem cell (HSC) compartment. It continues to be expressed during differentiation, decreasing with terminal differentiation, although it does continue through dendritic cell development. In disease, FLT3 mutations lead to constitutive kinase activity, activating multiple signaling pathways including STAT5, AKT and MAPK/ERK. These powerful signals in turn lead to the downstream effects that are the hallmarks of FLT3-mutant AML: differentiation blockade, inhibition of apoptosis, and a proliferative advantage.

FLT3 mutations occur in cooperation with mutations in many other genes, most frequently with DNMT3A and NPM1, and are often the last step leading to full transformation to AML. The most common FLT3 mutation is the internal tandem duplication (ITD) mutation. This mutation is characterized by the in-frame duplication of a small sequence of variable length within the FLT3 juxtamembrane domain (JMD). This leads to loss of autoregulation through the JMD and subsequent constitutive FLT3/ITD activity. As a disease-defining mutation, it has been shown to be a high-risk feature, associated with increased rates of refractory disease and relapse, and decreased overall survival. In many studies, patients with FLT3/ITD AML have shown a 15-25% event-free survival in response to standard AML chemotherapy protocols compared to 40-60% in patients without FLT3/ITD mutations.(3) As a result, FLT3-mutated AML has not benefited from many of the advances seen in outcomes for other leukemias and has required the additional treatment of bone marrow transplant with its short and long-term toxicities to improve outcome. In contrast to their chemoresistance, AML blasts with FLT3 mutations are preferentially sensitive to tyrosine kinase inhibitors (TKI) against FLT3, making FLT3 an attractive target for drug development.

Despite the promise of FLT3-directed therapy, clinical trials featuring FLT3 TKI have had limited success. Multiple factors have contributed to these outcomes including resistance mutations in FLT3, bone marrow stromal-derived survival factors, overexpression of FLT3 ligand, and activation of alternative “bypass” pathways. The study of these causes of failure has led to FLT3 TKI with ever-increasing efficacy. However, the role of patient-specific factors in modulating treatment efficacy remains relatively unexplored in the context of FLT3 AML research.

Prior in vitro and clinical trial data indicate that the lack of FLT3-specific clinical impact by tyrosine kinase inhibitors (TKI) therapy may be due, in part, to inadequate activity against FLT3 due to inhibition of these agents by patient-specific factors such as plasma protein binding, stromal protection and altered pharmacokinetics. Prior work has demonstrated that binding by plasma proteins, especially alpha-1-acid glycoprotein (AGP), significantly reduces and even abrogates the activity of these drugs due to their extremely high binding affinity for protein. There is a commercial need to identify drugs that inhibit AGP binding to chemotherapy agents such as TKI. Such a drug, when given to a patient, would keep an administered chemotherapy agent substantially free of AGP and allow the chemotherapy agent to effectively bind to its target, inhibit cancer, and enhance the quality of life for cancer patients.

### SUMMARY OF THE INVENTION

One embodiment of the present invention are methods of inhibiting one or more first pharmaceutical agent from binding to a plasma protein. The methods comprise the steps of administering to a subject a plasma protein-binding agent prior to, concurrently with, or after the administration of a one or more pharmaceutical agent that binds to a plasma protein in vivo. Suitable plasma protein binding agents that may be used in the methods of the present invention include Mifepristone, Piperidolate, Roxithromycin, Paroxetine, Bupivacaine, Trihexyphenidyl, Carvedilol, Denatonium, Nifedipine, Benzonatate, Oxybutynin, Tolterodine tartate, Ethopropazine, Triprolidine, Imatinib, Thioridazine, Quinidine gluconate, Phenothiazine, Loxapine, Mebeverine, Penbutol, Procyclidine, Eticlopride, Cyclobenzaprine, Triflupromazine, Benzydamine, Promazine, Dicyclomine, Clomipramine, Yohimbine, Alprenolol, Quinine, Dibucaine, Chlorpromazine, Asenapine, or Clindamycin, or a salt, solvate, or stereoisomer thereof, and/or a combination thereof. Chemical structures of these drugs are provided in FIG. 14. Examples of a pharmaceutical agents that bind to a plasma protein includes a tyrosine kinase inhibitor (TKI). Examples of tyrosine kinase inhibitors (TKI) include TT-3002, lestaurtinib, midostaurin, midostaurin, and sorafenib, staurosporine-derived TKI, anti-FLT3 agents, or a salt, solvate, or stereoisomer, and/or combinations thereof. Chemical structures of these drugs are provided in FIG. 2. A pharmaceutical agent that binds to a plasma protein, such as a TKI, is less effective of treating disease including acute myeloid leukemia (AML), refractory neoplasms, or multiple myeloma. Administering a plasma binding protein of the present invention in combination with a TKI enhances the effectiveness of TKI to treat disease, such as those diseases listed above, when compared to a reference subject who has not be provided with a plasma binding protein in combination with a TKI, or a pharmaceutical agent that binds to plasma binding proteins.

Another embodiment of the present invention includes methods of inhibiting one or more pharmaceutical agent from binding to a plasma protein and treating a disease. The methods comprise the steps of administering to a subject a plasma protein binding agent prior to, concurrently with, or after the one or more pharmaceutical agent is administered to a subject to treat a disease of the subject; binding of the plasma protein binding agent to a plasma protein so that the one or more pharmaceutical agent is unable to bind to the plasma protein; and treating the disease of the subject.

Another embodiment of the present invention are methods of releasing one or more first pharmaceutical agent bound to a plasma protein. The methods comprise the steps of administering to a subject a plasma protein-binding agent prior to, concurrently with, or after the administration of a one or more pharmaceutical agent that binds to a plasma protein in vivo.

Another embodiment of the present invention are methods of releasing one or more pharmaceutical agent bound to a plasma protein and treating a disease. The methods comprise the steps of administering to a subject a plasma protein binding agent prior to, concurrently with, or after administration of the one or more pharmaceutical agent administered to a subject to treat a disease of the subject; unbinding the one or more pharmaceutical agent bound to a plasma protein; treating the disease of the subject; and forming an unbound pharmaceutical agent that binds to a target.

Unless defined otherwise, all technical and scientific terms used herein have the meaning commonly understood by a person skilled in the art to which this invention belongs. The following references provide one of skill with a general definition of many of the terms used in this invention: Singleton et al., Dictionary of Microbiology and Molecular Biology (2nd ed. 1994); The Cambridge Dictionary of Science and Technology (Walker ed., 1988); The Glossary of Genetics, 5th Ed., R. Rieger et al. (eds.), Springer Verlag (1991); and Hale & Marham, The Harper Collins Dictionary of Biology (1991). As used herein, the following terms have the meanings ascribed to them below, unless specified otherwise.

By “activity” is meant the ability of a gene to perform its function such as FMS-like tyrosine kinase 3 (FLT3) as an oxidoreductase.

By “agent” is meant any small molecule chemical compound, antibody, nucleic acid molecule, or polypeptide, or fragments thereof.

By “AGP” is meant alpha-1-acid glycoprotein (AGP) modulated by two polymorphic genes. AGP is synthesized primarily in hepatocytes and has a normal plasma concentration between 0.6-1.2 mg/ml (1-3% plasma protein).

By “ameliorate” is meant decrease, suppress, attenuate, diminish, arrest, or stabilize the development or progression of a disease.

By “alteration” is meant a change (increase or decrease) in the expression levels or activity of a gene or polypeptide as detected by standard art known methods such as those described herein. As used herein, an alteration includes a 10% change in expression levels, preferably a 25% change, more preferably a 40% change, and most preferably a 50% or greater change in expression levels.

By “AML” is meant acute myeloid leukemia.

By “analog” is meant a molecule that is not identical, but has analogous functional or structural features. For example, a polypeptide analog retains the biological activity of a corresponding naturally-occurring polypeptide, while having certain biochemical modifications that enhance the analog's function relative to a naturally occurring polypeptide. Such biochemical modifications could increase the analog's protease resistance, membrane permeability, or half-life, without altering, for example, ligand binding. An analog may include an unnatural amino acid.

By “disease” is meant any condition or disorder that damages or interferes with the normal function of a cell, tissue, or organ. Examples of diseases include cancer.

By “effective amount” is meant the amount of a required to ameliorate the symptoms of a disease relative to an untreated patient. The effective amount of active compound(s) used to practice the present invention for therapeutic treatment of a disease varies depending upon the manner of administration, the age, body weight, and general health of the subject. Ultimately, the attending physician or veterinarian will decide the appropriate amount and dosage regimen. Such amount is referred to as an “effective” amount.

The term “express” refers to the ability of a gene to express the gene product including for example its corresponding mRNA or protein sequence (s).

By “FLT3” is meant FMS-like tyrosine kinase 3 a protein produced from an FLT3 gene. FLT3 is part of a family of proteins called receptor tyrosine kinases (RTKS). Receptor tyrosine kinases transmit signals from the cell surface into the cell through a process called signal transduction.

By “fragment” is meant a portion of a polypeptide or nucleic acid molecule. This portion contains, preferably, at least 10%, 20%, 30%, 40%, 50%, 60%, 70%, 80%, or 90% of the entire length of the reference nucleic acid molecule or polypeptide. A fragment may contain 10, 20, 30, 40, 50, 60, 70, 80, 90, or 100, 200, 300, 400, 500, 600, 700, 800, 900, or 1000 nucleotides or amino acids.

The term, “obtaining” as in “obtaining an agent” includes synthesizing, purchasing, or otherwise acquiring the agent.

The terms “polypeptide,” “peptide” and “protein” are used interchangeably herein to refer to a polymer of amino acid residues. The terms apply to amino acid polymers in which one or more amino acid residue is an analog or mimetic of a corresponding naturally occurring amino acid, as well as to naturally occurring amino acid polymers. Polypeptides can be modified, e.g., by the addition of carbohydrate residues to form glycoproteins. The terms “polypeptide,” “peptide” and “protein” include glycoproteins, as well as non-glycoproteins.

By “Range” or “Ranges” is meant to be shorthand for all of the values within the range. For example, a range of 1 to 50 is understood to include any number, combination of numbers, or sub-range from the group consisting 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50.

By “reduces” is meant a negative alteration of at least 10%, 25%, 50%, 75%, or 100%.

By “reference” is meant a standard or control conditions such as a sample (human cells) or a subject that is a free, or substantially free, of an agent such as a plasma protein binding agent of the present invention.

By “specifically binds” is meant an agent that recognizes and binds a polypeptide or protein such as a plasma protein such as alpha-1-acid glycoprotein (AGP) for example, but which does not substantially recognize and bind other polypeptides or proteins in a sample, for example, a biological sample, which naturally includes a polypeptide or protein described in the invention.

By “subject” is meant any individual or patient to which the method described herein is performed. Generally, the subject is human, although as will be appreciated by those in the art, the subject may be an animal. Thus other animals, including mammals such as rodents (including mice, rats, hamsters and guinea pigs), cats, dogs, rabbits, farm animals including cows, horses, goats, sheep, pigs, etc., and primates (including monkeys, chimpanzees, orangutans and gorillas) are included within the definition of subject.

By “TKI” is meant Tyrosine Kinase Inhibitor, a pharmaceutical agent that inhibits tyrosine kinases. Tyrosine kinases, including FLT3, are enzymes responsible for the activation of many proteins by signal transduction cascades. The proteins are activated by adding a phosphate group to the protein, a step that TKIs inhibit.

By “treat,” “treating,” “treatment,” and the like is meant to reducing or ameliorating a disorder and/or symptoms associated therewith. It will be appreciated that, although not precluded, treating a disorder or condition does not require that the disorder, condition or symptoms associated therewith be completely eliminated.

Unless specifically stated or obvious from context, as used herein, the term “or” is understood to be inclusive. Unless specifically stated or obvious from context, as used herein, the terms “a”, “an”, and “the” are understood to be singular or plural.

Unless specifically stated or obvious from context, as used herein, the term “about” is understood as within a range of normal tolerance in the art, for example within 2 standard deviations of the mean. About can be understood as within 10%, 9%, 8%, 7%, 6%, 5%, 4%, 3%, 2%, 1%, 0.5%, 0.1%, 0.05%, or 0.01% of the stated value. Unless otherwise clear from context, all numerical values provided herein are modified by the term about.

The recitation of a listing of chemical groups in any definition of a variable herein includes definitions of that variable as any single group or combination of listed groups. The recitation of an embodiment for a variable or aspect herein includes that embodiment as any single embodiment or in combination with any other embodiments or portions thereof.

Any compositions or methods provided herein can be combined with one or more of any of the other compositions and methods provided herein.

As used herein, the terms “prevent,” “preventing,” “prevention,” “prophylactic treatment” and the like refer to reducing the probability of developing a disorder or condition in a subject, who does not have, but is at risk of or susceptible to developing a disorder or condition.

Such treatment (surgery and/or chemotherapy) will be suitably administered to subjects, particularly humans, suffering from, having, susceptible to, or at risk for cancer or disease, disorder, or symptom thereof. Determination of those subjects “at risk” can be made by any objective or subjective determination by a diagnostic test or opinion of a subject or health care provider (e.g., genetic test, enzyme or protein marker, a marker (as defined herein), family history, and the like).

### BRIEF DESCRIPTION OF THE DRAWINGS

FIG. 1: FLT3 inhibitors are differentially inhibited by human plasma and AGP. Co-incubation with human plasma or human AGP inhibits FLT3 TKI activity. (A) Proliferation of MOLM-14 cells after 48 hours of TKI exposure in the presence or absence of 50% human plasma was assessed by colorimetric methods (MTT). (B) MOLM-14 cells were exposed to low (10 nM) or high (100 nM) concentrations of TKI as well as media (10% FBS), 50% human plasma or human AGP and analyzed by phospho-Western blotting for FLT3 phosphorylation and downstream signaling. Apoptosis was measured by (C) flow cytometry (Annexin V and 7-AAD staining) or (D) Western analysis for cleaved PARP after 24 hours of TKI exposure under standard culture conditions (10% FBS) or in the presence of either 100% human plasma or purified human AGP. (E) Proliferation and (F) Western analysis experiments were repeated in the presence of 50% FBS, bovine plasma, or purified bovine AGP and compared to comparable results with the corresponding human fractions.

FIG. 2: FLT3 inhibitors show variable inhibition by human plasma in both AGP-dependent and independent fashions. MOLM-14 cells were exposed to increasing concentrations of the indicated FLT3 TKI under standard culture conditions (10% FBS) or in the presence of either (A) 50% human plasma or (B) various concentrations of purified human AGP (0.25 mg/mL, 0.5 mg/mL, 1.0 mg/mL), and proliferation was measured by MTT after 48 hours. (C) Structures of the various FLT3 TKI used.

FIG. 3: AGP demonstrates linear, dose-dependent inhibition of FLT3 TKI. (A) MOLM-14 cells were co-cultured in a range of concentrations of AGP (31 μg/mL-2 mg/mL) and treated with increasing doses of the FLT3 TKI, TTT-3002. Proliferation was measured by MTT after 48 hours. (B) The IC50 for TTT-3002 was plotted as a function of AGP concentration. (C) Plasma from 19 human patients was assayed for the ability to inhibit TTT-3002 using the modified PIA against MOLM-14 cells. The fold-change in the IC50 (relative to cells treated under standard 10% FBS culture conditions) was calculated and plotted as a function of plasma AGP concentration as measured by radial immunodiffusion assay. Linear regression, best fit is plotted (solid line) with the curve generated in (B) also plotted for comparison (dashed line). By convention, patient AGP concentration is reported as mg/dL.

FIG. 4: Human AGP binds staurosporine-derived FLT3 TKI with variable affinities. (A) MOLM-14 cells were co-cultured in a range of concentrations of human AGP and treated with various staurosporine-derived FLT3 TKI. The fold-change in the IC50 (relative to cells treated under standard 10% FBS culture conditions) was calculated and plotted as a function of free AGP concentration (corrected for bound drug: IC50 with AGP—IC50 with 10% FBS). (B) AGP binding constants for each drug were calculated from the linear regions of each curve (solid line); see text and supplemental materials for mathematical treatment. (C) Scatchard plots for fluorescence displacement assays examining competitive displacement of ANS from AGP by the indicated drugs.

FIG. 5: Inhibition of drug-protein binding is dependent upon protein and protein binding inhibitor characteristics and concentrations. (A) Key aspects of the fold-change growth inhibition curves generated using an idealized drug (KA=10 μM-1) and a binding inhibitor (KI=10 μM-1, I0=10 μM). Growth inhibition curves are shown for both the presence (orange) and absence (blue) of a binding inhibitor. Shown are the curves and equations for the asymptotes at low (green) and high (red) protein concentrations, the apparent free-protein concentration (purple) at which the curve has the greatest rate of increasing slope and the maximal shift (bracket) in the curve caused by the presence of a binding inhibitor. (B) The effect was experimentally measured in MOLM-14 cells as the fold-change in IC50 of lestaurtinib for cells co-cultured with varying concentrations of AGP and mifepristone, a potential binding inhibitor. Mifepristone concentrations: 15 μM, 10 μM, 5 μM, AGP concentrations: 2-fold dilutions from 1 mg/mL to 3.9 μg/mL (1 mg/mL≈20 μM AGP). FIG. 6: AGP inhibition of TKIs can be abrogated by the addition of competitor compounds. (A) Competitive fluorescence displacement was performed in the presence of increasing concentrations of AGP-binding drugs (Lestaurtinib, Midostaurin, and Mifespristone). ANS only exhibits fluorescence when bound to AGP. (B) Dose-response curves using the modified PIA in the presence of increasing doses of mifepri stone show a return of the curves towards those seen under standard culture conditions (10% FBS) despite the presence of inhibitory levels of AGP. (C) Co-incubation of MOLM-14 cells with mifepristone restores TTT-3002 activity (5 nM) in the presence of AGP in a mifepristone dose-dependent manner. (D) MOLM-14 cells were treated with TTT-3002 (2 nM) in the presence or absence of inhibitory levels of AGP and the effect of co-incubation with mifepristone upon FLT3/ITD signaling inhibition was measured through phospho-Western analysis.

FIG. 7: Identification of compounds that can restore TKI activity despite inhibitory levels of AGP. (A) Ba/F3-FLT3/ITD cells were exposed to cytotoxic concentrations of midostaurin (100 nM) in the presence of inhibitory levels of AGP (0.5 mg/mL) and co-cultured with a library of 2,800 FDA-approved drugs with or without 1 ng/mL of IL-3. After 48 hours in culture, cell growth was assessed by the MTT colorimetric assay and growth in IL-3 was plotted against growth in its absence. Candidate compounds are those that show cytotoxicity that can be completely (area 1) or partially (area 2) rescued by IL-3. (B) Compounds were also measured for their ability to displace AGP-bound ANS, and IL-3-rescuable growth inhibition was plotted as a function of ANS binding to AGP. Compounds that both displace ANS from AGP and restore IL-3-rescuable cytotoxicity are enclosed in the red dashed region. (C) Validation of one candidate compound. Its position on plots figures A and B is indicated in the inserts.

FIG. 8: Human AGP inhibits midostaurin efficacy and can be rescued by mifepristone in vivo. (A) Experimental scheme. (B) Human AGP was injected intraperitoneally at 0 and 48 hours and the plasma concentration was measured at various timepoints via human-specific ELISA. Green line indicates the average of 4 independent mice (gray lines). (C) Sub-lethally irradiated BALB/c mice were engrafted with 5×105 Ba/F3-FLT3/ITD-luc cells. Bioluminescent imaging on day 5 confirmed engraftment. After engraftment, recipients received human AGP or vehicle by IP injection, and either midostaurin or vehicle by oral gavage for four days. A sub-group of hAGP/midostaurin mice also received mifepristone by oral gavage. Response was assessed on day 9 by repeat bioluminescent imaging. Shown is a representative example of duplicate runs.

FIG. 9: TKI-induced cell cycle arrest is inhibited by AGP. Cells were cultured under either standard culture conditions (10% FBS) or with 0.5 mg/mL AGP in the presence of low (10 nM) or hight (100 nM) concentrations of TTT-3002 or CEP-701. After 24 hours of exposure, the cells were stained with propidium iodide and analyzed for cell cycle distribution flow cytometry. Populations were imputed using standard algorithms (FlowJo).

FIG. 10: FLT3 inhibitors bind AGP with differential affinity. Demonstration of principle for the highly inhibited CEP-701. MOLM-14 cells were co-cultured in a range of concentrations of AGP (31 μg/mL-2 mg/mL) and treated with increasing doses of the FLT3 TKI, CEP-701. Proliferation was measured by MTT after 48 hours.

FIG. 11: TTT-3002 has the greatest predicted in vivo anti-FLT3 activity despite AGP binding. MOLM-14 cells were co-cultured in a range of concentrations of human AGP and treated with various staurosporine-derived FLT3 TKI. The IC50 was calculated and plotted as a function of AGP concentration. The predicted in vivo IC50 (based upon an average AGP plasma concentration of 100 mg/dL) was extrapolated and shown in the table insert.

FIG. 12: Modeling two-drug competition for AGP. (A) Protein-binding associated fold-change increases for drug with a binding constant of KA=10 μM-1 was calculated in the presence of a protein-binding inhibitor. Shown are the response curves plotted as a function of free-protein concentration (μM). Shown is the control simulation of no inhibitor (I0=0 μM), and the addition of an inhibitor of equal affinity with a concentration of the same (I0=10 μM, KI=10 μM-1). Simulated was the effects of increasing (I0=20 μM) and decreasing (I0=5 μM) the concentration of the inhibitor while maintaining the same affinity (KI=10 μM-1), as well as increasing (KI=40 μM-1) or decreasing (KI=2 μM-1) the affinity of the inhibitor while maintaining the same concentration (I0=10 μM). (B) Competition of an idealized drug (KA=10 μM-1) and inhibitor (KI=10 μM-1, I0=10 μM) pair for binding to a protein was calculated using an iterative, perturbation method. For each cycle, the active drug (Af) was set as 10 nM, and the bound drug (PA) was calculated based upon the mass action equilibrium for drug-protein binding alone to determine total drug necessary (A0), fold-change (Δ), and apparent free-protein concentration (t). These results were then used to perturb the inhibitor-protein equilibrium, which in turn determined the amount of protein available for drug binding in the next cycle. The cycle was repeated at least 40 times until convergence (error≤10-16-fold) of Δ was obtained. Plotted are simulations for P0=20 μM, 15 μM, 10 μM, 5 μM, 2.5 μM and 0 μM. The exact curves obtained from equation 18 were plotted for comparison. For each simulation, the results differed from the calculated fold-change by less than 10-14-fold.

FIG. 13: Lower-affinity compounds cannot displace the more tightly-bound TKI from AGP. Dose-response curves for TTT-3002 (left panels) and CEP-701 (right panels) were generated from MOLM-14 cells using the modified PIA in the presence or absence of AGP (1.0 mg/mL) co-incubated with increasing doses of celecoxib (A), thalidomide (B) or amitriptyline (C), all of which have been reported to bind to AGP with various affinities.

FIG. 14: List of Plasma Protein Binding Agents.

### DETAILED DESCRIPTION OF THE INVENTION

The inventors have performed research and determined the impact of plasma protein binding on FLT3 TKI activity, focusing on the staurosporine family of which the recently-approved drug midostaurin (Rydapt, PKC-412) is a member. Furthermore, the inventors have discovered a novel method for overcoming plasma protein inhibition to restore the promise of molecularly targeted therapy, including FLT3 mutated AML as well as for other TKI whose activity is diminished by protein binding.

Human Plasma and Plasma Proteins Reduce TKI Efficacy

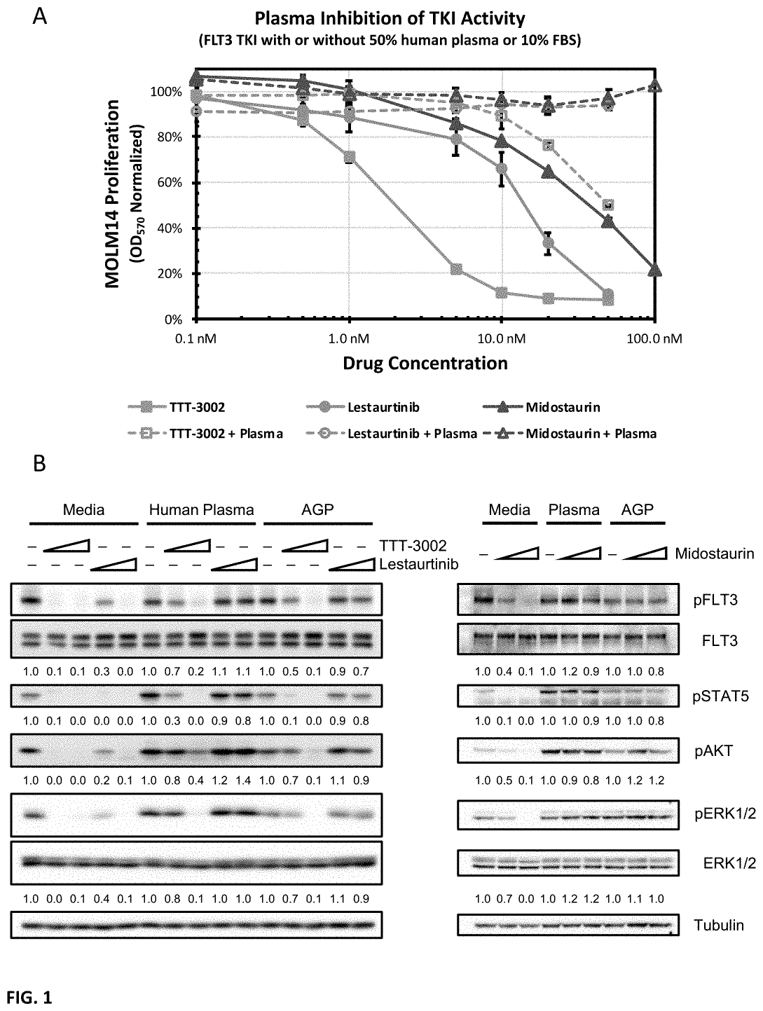

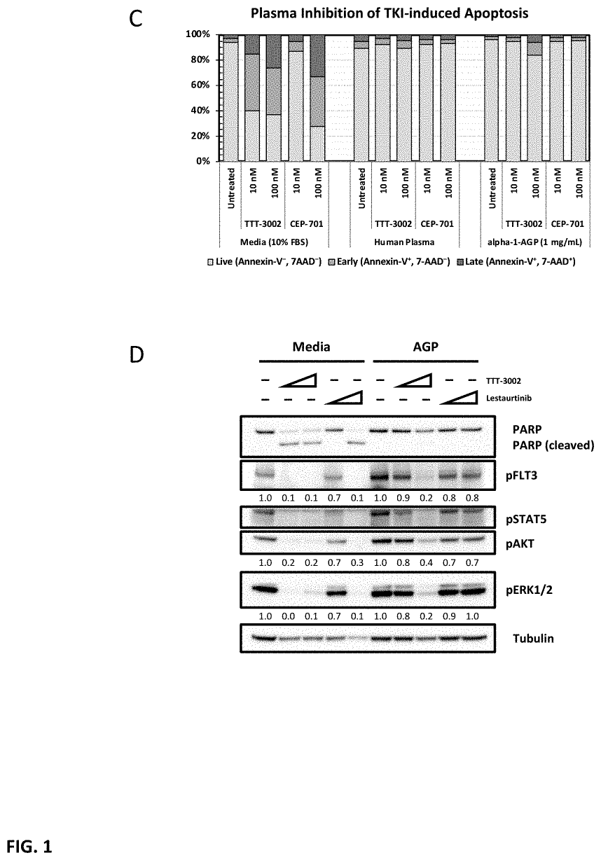

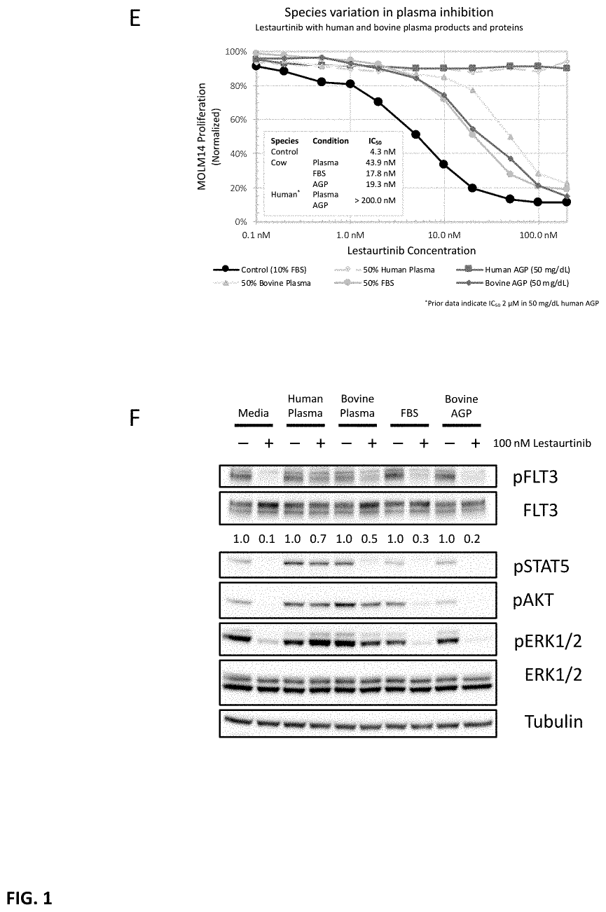

In the original plasma inhibitory assay (PIA), FLT3/ITD-expressing cells are co-incubated with plasma samples from patients receiving a FLT3 TKI, and Western blotting analysis of FLT3 auto-phosphorylation is used to measure the extent of FLT3 inhibitory activity present in their plasma.(9) This can then be compared to total plasma drug levels to determine the relative efficacy of a drug in vivo compared to its activity in vitro. From these results, it is possible to impute the effects of host factors on drug efficacy beyond simple metabolism. To model these effects in the pre-clinical setting, we modified the PIA assay. Whereas the PIA was designed to assess in vivo drug activity with regards to a specific target (e.g.: inhibition of FLT3 auto-phosphorylation by TKI), the modified PIA is similarly cell-based, but measures the clinically relevant biological end-points of cytotoxicity and proliferation inhibition, instead of target inhibition. In the modified assay, FLT3/ITD cell lines are cultured in the presence of human plasma or purified human plasma proteins and treated with a range of concentrations of the drug of interest. The IC50 measured using the colorimetric MTT method is compared to treatment under typical in vitro culture conditions of 10% fetal bovine serum (FIG. 1A). Furthermore, like the original PIA, Western blot analysis can be performed on cells treated in this manner to study specific pathways of interest (FIG. 1B). Thus, it is possible to simulate, in vitro, the in vivo effect of plasma proteins upon drug activity, allowing one to investigate the effect of endogenous proteins in their native state upon drug dose-response relationships in a controlled manner without the confounding variables of patient-specific drug bioavailability and metabolism. The modified PIA utilizes the general endpoint of cytotoxicity. Therefore, it is readily adaptable to other disease types and agents where plasma protein binding has been implicated in affecting therapeutic efficacy.

Using this assay, the inventors treated MOLM-14 cells (FLT3/ITD heterozygous, KMT2A-rearranged myeloid leukemia cell line) for 48 hours with midostaurin, lestaurtinib or TTT-3002 across a range of physiologically relevant concentrations in the presence of either 50% human plasma from healthy donors or standard 10% fetal bovine serum culture conditions (FIG. 1A). For all three TKI, the addition of human plasma significantly increased the IC50 of each drug, and for midostaurin and lestaurtinib, IC50s were no longer reached in the range of the conditions tested.

To demonstrate that this effect is through reduced FLT3 inhibition, we treated MOLM-14 cells for 1 hour with each TKI at concentrations greater than the IC50 for inhibition of FLT3 autophosphorylation (10 nM and 100 nM) and assessed FLT3 autophosphorylation and downstream signaling in the presence or absence of human plasma (FIG. 1B). For TTT-3002, the presence of human plasma lead to a significant decrease in inhibition of FLT3 phosphorylation, and subsequent downstream decreased inhibition of phosphorylation/activation of STAT5, AKT1 and ERK1/2. While this effect was overcome by increasing the concentration of TTT-3002, for midostaurin and lestaurtinib, there was still no appreciable FLT3 TKI inhibitory activity in the presence of plasma even at a ten-fold increase in drug concentration. Previous work and the results of clinical trials with FLT3 TKI have suggested that alpha-1-acid glycoprotein (AGP), an acute phase acid glycoprotein responsible for scavenging basic drugs and steroids, plays a role in binding to these FLT3 TKI.(10-16) When these experiments were repeated in the presence of physiologically equivalent concentrations of purified AGP (1 mg/mL) instead of whole human plasma, comparable inhibition of anti-FLT3 activity and associated downstream disinhibition was again observed (FIG. 1B).

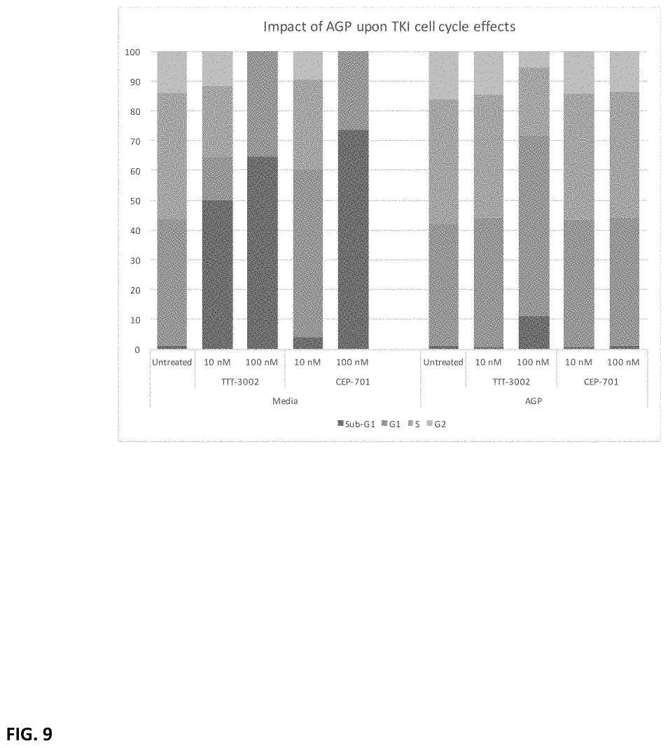

The inventors repeated the modified PIA, instead assaying for apoptosis after 24 hours, and again found that the presence of human plasma abrogates TKI-induced apoptosis (FIG. 1C) and associated PARP cleavage (FIG. 1D). Again, the use of physiologic concentrations of human AGP in place of whole plasma is sufficient to reproduce this effect (FIG. 1C). These results were also seen in the TKI-mediated effects on cell cycle analysis (FIG. 9). The addition of human AGP alone does not increase FLT3 signaling, alter baseline apoptosis, or change cell cycle distribution. Taken together, these data indicate that human AGP plays a significant role in the observed plasma inhibition of TKI activity, presumably through binding and sequestering the drugs away from their cellular target. Furthermore, when these experiments are repeated using bovine plasma or bovine AGP, there is little effect on the activity of FLT3 TKI (FIG. 1E, 1F). These results, along with the lower expression of AGP in mice and cow, likely explain why these effects have not been observed in pre-clinical in vitro and in vivo murine studies.

### FLT3 TKI are Differentially Affected by Human Plasma and Purified Human Plasma Proteins

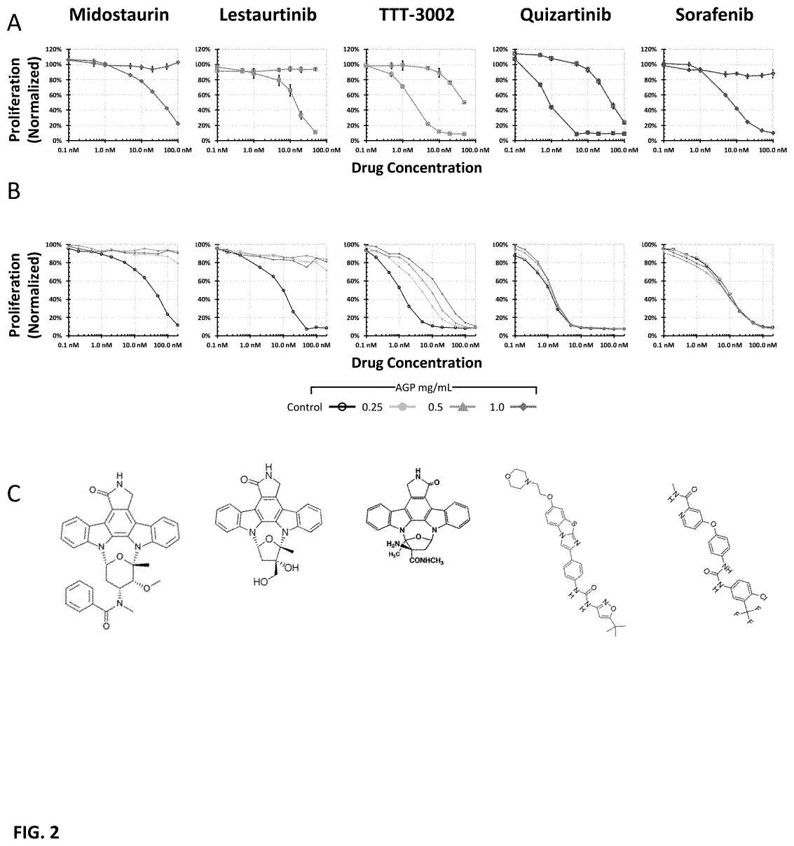

The inventor's tested a panel of FLT3 TKI for activity in the modified PIA. All of the TKI tested demonstrated an increase in IC50 when tested in the presence of 50% human plasma (FIG. 2A). These ranged from minimal (9-fold) inhibition for TTT-3002 to moderate inhibition (37-fold) for quizartinib and to greater than 100-fold inhibition for lestaurtinib, midostaurin, and sorafenib. When this panel was tested using purified human AGP at physiologic concentrations (1 mg/mL), quizartinib and sorafenib activity were not significant inhibited, while TTT-3002 demonstrated comparable inhibition to that observed with human plasma (FIG. 2B). A complete inhibition of lestaurtinib and midostaurin activities were observed, even at sub-physiologic concentrations. Examination of structures show that the TKI affected significantly by AGP (lestaurtinib, midostaurin, TTT-3002) are all derivatives of staurosporine, in contrast to those unaffected by AGP (FIG. 2C).

### Plasma AGP Concentration is the Primary Determinant of Plasma Protein Inhibition of Staurosporine-Derived TKI

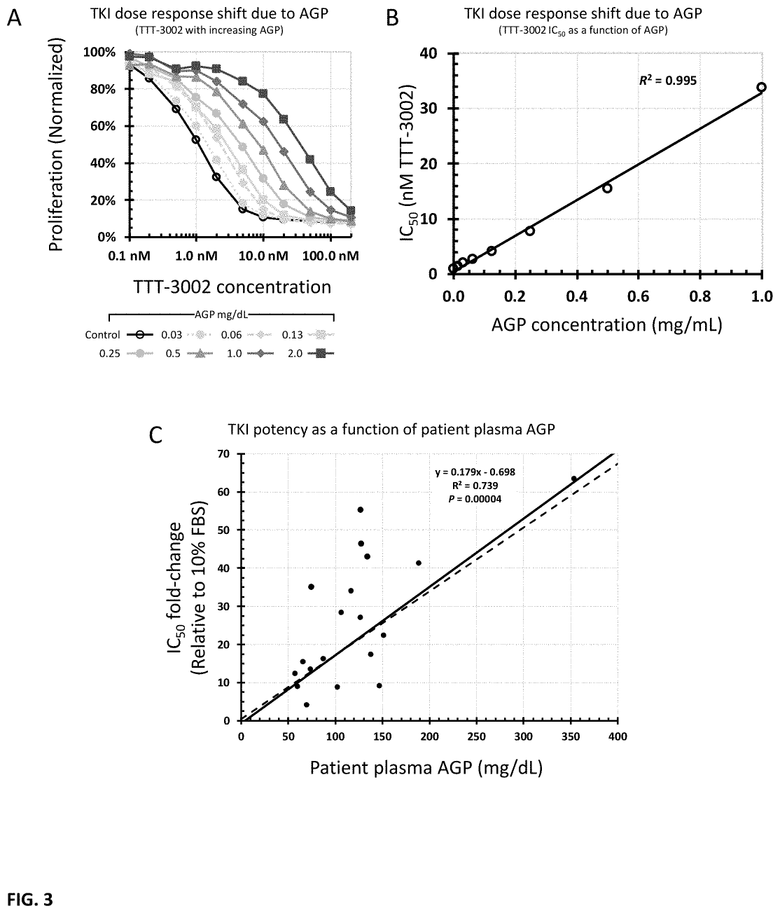

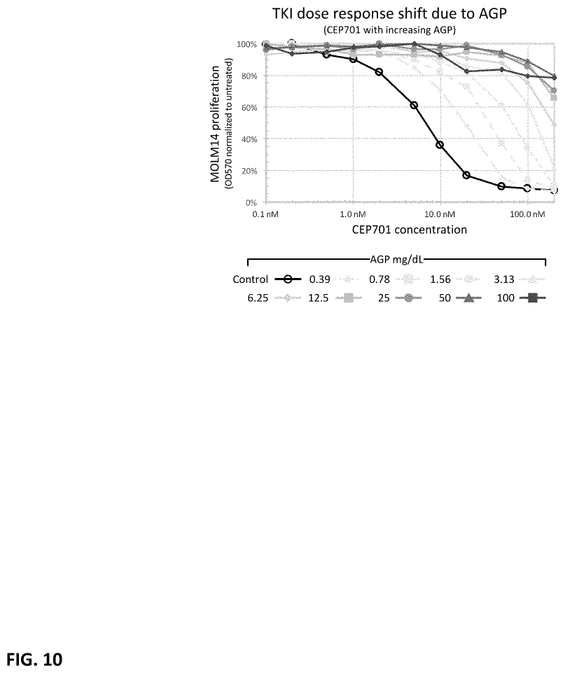

To better characterize the impact of human plasma on the staurosporine-derived TKI, the inventors examined the relationship between fold-change in the IC50 of TTT-3002 and AGP concentration. When the modified PIA was performed for TTT-3002 using purified human AGP across a range of concentrations, a relationship was seen between AGP concentration and the associated TTT-3002 dose-response curves (FIG. 3A). Measuring IC50 at each AGP concentration and comparing it to the IC50 observed under standard culture conditions (IC50 fold-change), a direct linear relationship was noted between the fold-change in IC50 and AGP concentration (FIG. 3B). When these studies were conducted using lestaurtinib, a similar relationship was noted. Of note, appreciable lestaurtinib activity is only seen at AGP concentrations less than one-tenth of typical physiologic human levels (FIG. 10).

As these results suggest that the major determinant of plasma inhibition of staurosporine TKI is AGP, we studied the relationship of plasma AGP concentration to plasma inhibition. Plasma samples from 19 human donors was collected. AGP concentration was measured, and the plasma was tested for TKI inhibition using the modified PIA for TTT-3002. The results showed a linear correlation between plasma AGP concentration in the samples and the resulting fold-change in observed IC50 (FIG. 3C, solid line) which correlated closely to the results observed using purified AGP (FIG. 3C, dashed line). Analysis of other factors (total protein concentration, albumin concentration, platelet count, and hemoglobin concentration) did not demonstrate any significant correlation (data not shown).

### The Modified Plasma Inhibition Assay Measures TKI-AGP Affinity

We examined the relationship of IC50 fold-change and plasma protein concentration, and demonstrated:

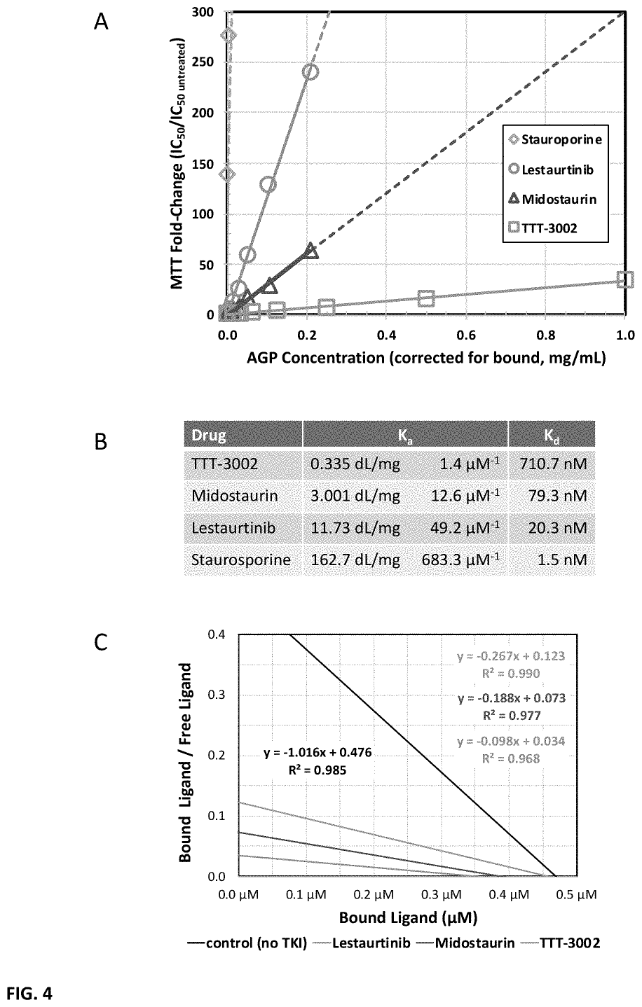

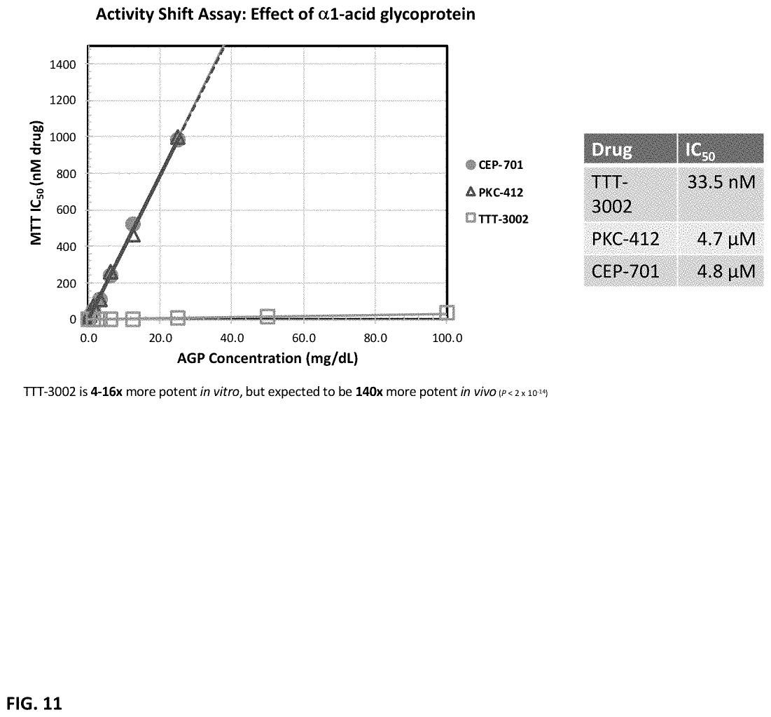

Δ=K_{A}(P₀−P_{A})+1

Where Δ is the fold-change in IC₅₀, K_{A} is the drug-protein association constant, P₀ is the total protein concentration and P_{A} is the drug-bound protein concentration. From this relationship, in the absence of other drug-protein interactions, fold-change is linearly related to drug-free protein concentration. From this equation, it is apparent that comparing fold change in IC₅₀ to plasma AGP (FIG. 3A) yields biochemical data about the interaction of TKI with their associated plasma protein. When the analysis also takes into account drug-bound protein, these curves can be used to directly measure K_{A}. To that end, we performed the modified PIA using purified AGP against the staurosporine-derived FLT3 TKI, using MOLM-14 cells (FIG. 4A). For comparison, we also assayed the anti-proliferative effects of non-specific protein kinase C inhibition by staurosporine itself in HL-60 cells (FLT3 wild-type AML). The different TKI show variable sensitivity to AGP inhibition (P<2×10⁻¹⁶) with TTT-3002 being the least impacted (30-fold inhibition at physiologic concentrations) midostaurin showing intermediate inhibition (300-fold inhibition) and lestaurtinib showing the highest (>1000-fold inhibition predicted). In comparison, staurosporine demonstrated no appreciable activity in the presence of AGP until saturating concentrations of drug were used. Using regression analysis on the linear areas of these curves, we were able to calculate binding constants for each drug (FIG. 4B). Of note, although midostaurin shows only intermediate levels of inhibition, when correcting for lower potency for FLT3 inhibition compared to lestaurtinib, the two drugs are predicted to have approximately the same in vivo activity (FIG. 11).

To validate this approach as a quantitative assay, we also performed traditional competitive fluorescence displacement assays using 8-anilinonaphthalene 1-sulfonic acid (ANS), a polyaromatic compound that only fluoresces when bound to AGP. By measuring ANS fluorescence across a range of concentrations with fixed AGP, in the presence or absence of a competing drug of interest and then performing Scatchard analysis, we were able to use the Cheng-Prusoff approximation to measure drug-AGP affinity (FIG. 4C). These results were comparable to the results of the cell-based approach.

Overcoming Plasma Protein Inhibition with Mifepristone

The above considerations are valid only in the absence of competitive drug-protein interactions. When we expanded our analysis to include a competitive binder (I) of AGP, we found that the fold-change/protein concentration relationship can be formulated as:

$$Δ = \frac{{K}_{A}}{2 {K}_{I}} ( β - γ + \sqrt{{( β - γ )}^{2} + 4 β} ) + 1$$

### Where:

γ=K_{I}I₀+1

β=K_{I}τ

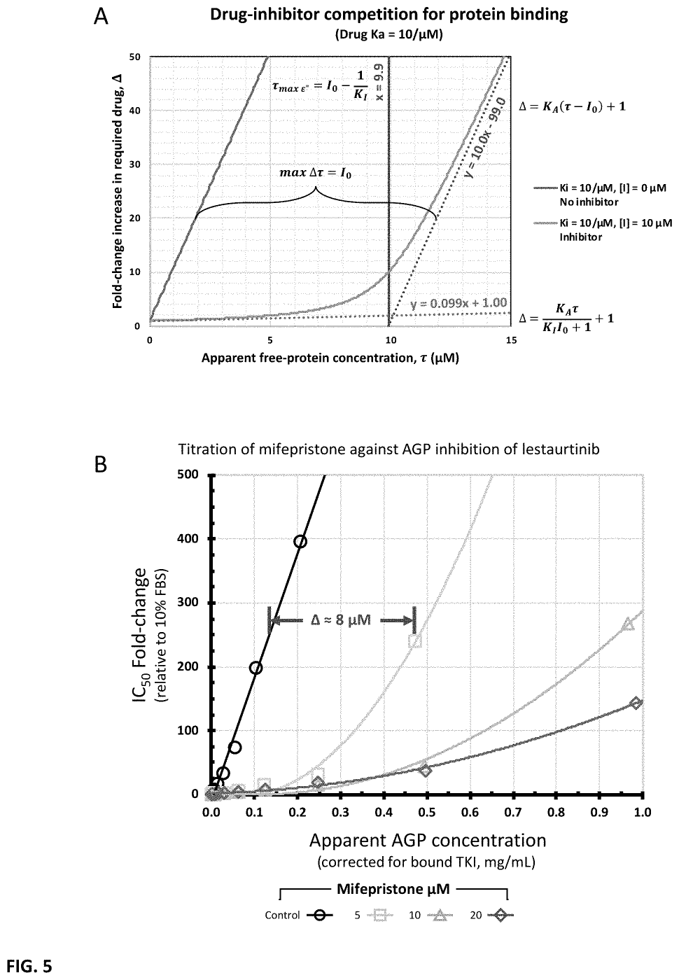

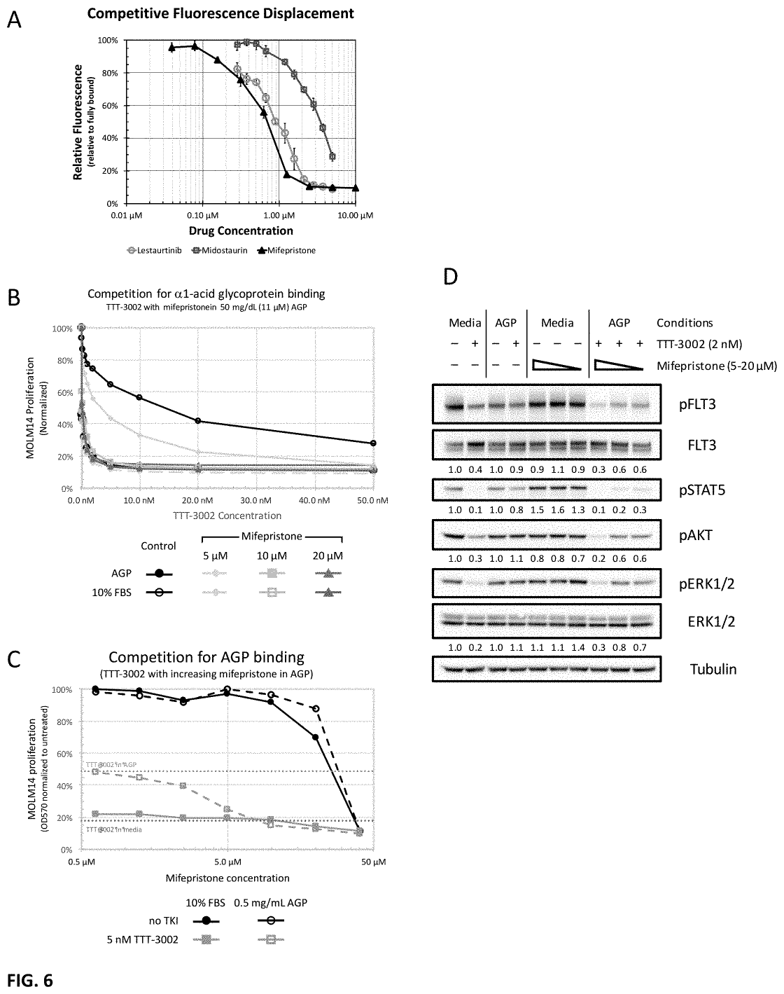

This relationship demonstrates that if the inhibitor binds with comparable affinity to AGP, it will have a significant impact upon the fold-change, reducing the overall inhibition of the drug mediated by protein-binding (FIG. 5).

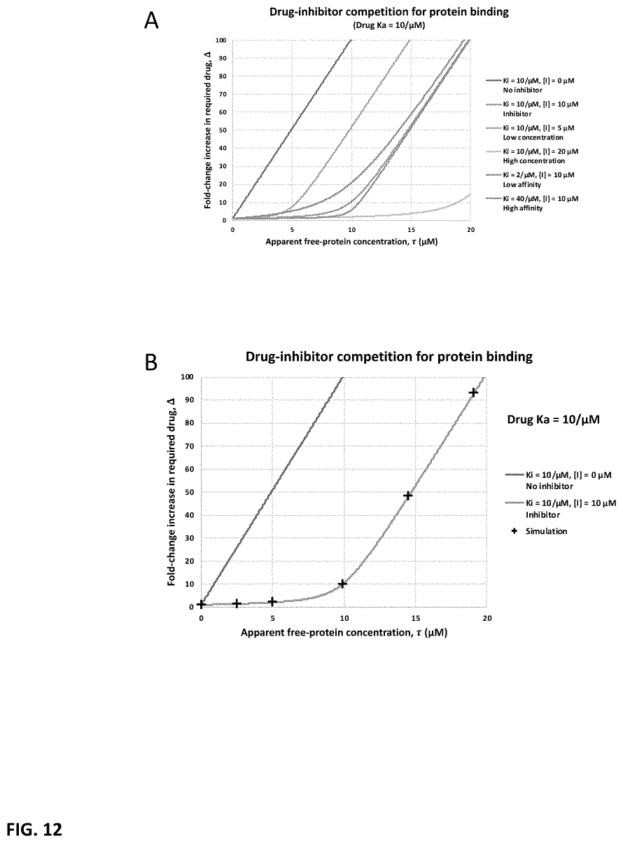

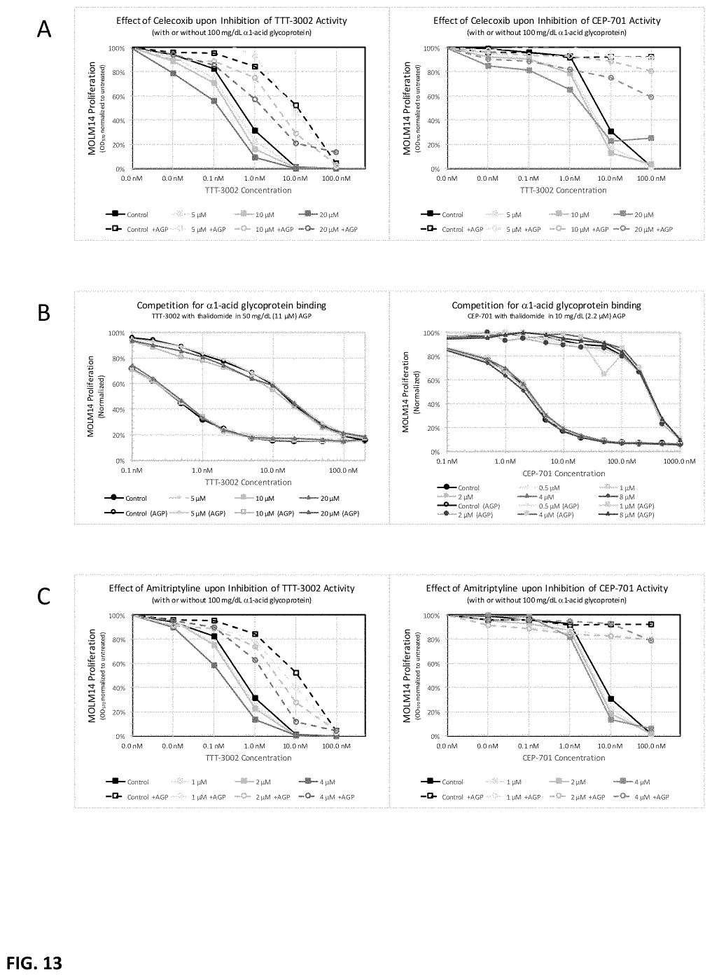

Given the above considerations, we undertook a proof-of-principle demonstration of disinhibiting FLT3 TKI using unrelated drugs to competitively bind AGP and displace the TKI. Of the known AGP-bound drugs, the steroid-derived, estrogen receptor antagonist, mifepristone has one of the highest association constants reported. With a K_{A} of 7-8 μM⁻¹, it is predicted to bind to AGP with higher affinity than TTT-3002 and comparable affinity to lestaurtinib and midostaurin. This is confirmed by competitive fluorescence displacement wherein mifepristone is able to displace ANS from AGP at concentrations 1.5 to 2-fold lower than midostaurin and lestaurtinib (FIG. 6A). Consequently, when we performed the modified PIA using TTT-3002 and purified AGP in the presence of increasing concentrations of mifepristone, we found that the dose-response curve shifted back to that seen in the absence of AGP (FIG. 6B). Mifepristone had no impact upon the TTT-3002 dose-response curves in the absence of AGP. Furthermore, when we titrated mifepristone in the presence or absence of AGP and TKI, we found that growth inhibition of MOLM-14 cells by TKI in the presence of AGP was returned to that seen in the absence of AGP at concentrations of mifepristone comparable to those achieved in clinical trials (FIG. 6C). The maximal effect occurs at concentrations where mifepristone alone has no appreciable effect on cell growth. Similar effects were not seen using other known AGP-binding agents with lower affinities (FIG. 12). These effects (FIG. 5B) agree within experimental limits to the model described above (FIG. 5A).

When MOLM-14 cells are treated with TTT-3002 in the presence of inhibiting concentrations of AGP, FLT3 inhibition is lost and de-repression of downstream signaling is observed. When cells are treated with mifepristone alone, there is no effect upon FLT3 autophosphorylation or downstream signaling. However, the addition of mifepristone at increasing concentrations results in a dose-dependent restoration of FLT3 inhibition by TTT-3002 with loss of FLT3-dependent downstream signaling (FIG. 6D). This provides further support that mifepristone is acting through competitive displacement of TKI from AGP and not through off-target effects.

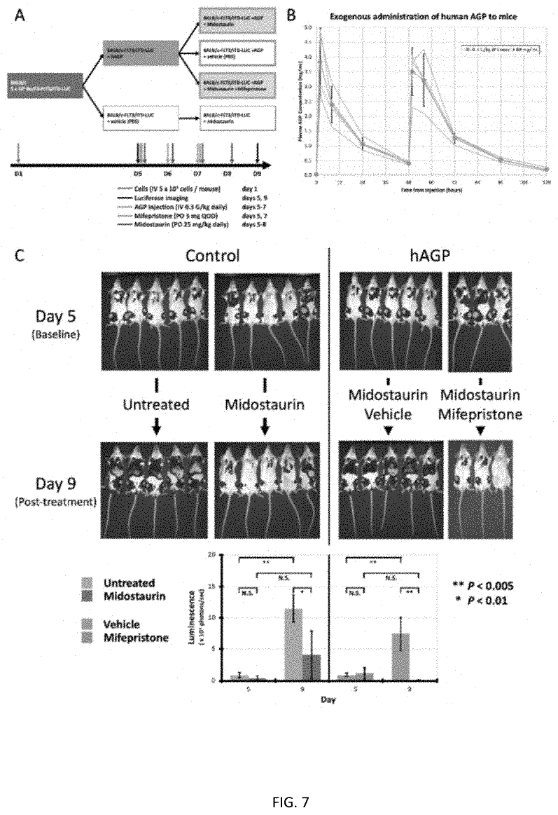

The inventors hypothesized that among the various FDA-approved drugs and supplements, there might exist a subset of agents that, much like mifepristone, are able to bind AGP and displace bound TKI and restore anti-FLT3 activity. Such agents might prove to be useful as combinatorial agents in restoring clinical activity of the staurosporine derivatives or serve as lead compounds for the development of such agents. To this end, we screened the Johns Hopkins Drug Library (JHDL), a collection of 2,560 different agents approved for human or veterinary use by the FDA. FLT3/ITD-expressing Ba/F3 cells were co-cultured in cytotoxic concentrations of midostaurin (100 nM) with inhibiting levels (0.5 mg/mL) of human AGP. To these we added the various agents of the JHDL at 20 μM and measured cell growth by colorimetric means. To confirm that any cytotoxicity was FLT3-specific, and not a consequence of off-target activity by the JHDL compounds themselves, we performed parallel platings in the presence or absence of recombinant murine IL-3 (IL-3). The IL-3 (on which the cells are normally dependent for their growth) should rescue the cells from FLT3-specific cytotoxicity but not from off-target cytotoxicity. The majority of compounds had no impact upon cell growth or were equally cytotoxic in the presence or absence of IL-3 (FIG. 7A). However, we identified 219 different agents that showed less cytotoxicity in the presence of IL-3. Although approximately 30 compounds were still significantly cytotoxic in the presence of IL-3 (FIG. 7A, area 2), the cytotoxicity of many of the compounds was mostly IL-3-reversible (area 1), indicating a subset of compounds that may act by displacing midostaurin from AGP and restoring its anti-FLT3 activity.

To further screen these agents, we also performed competitive fluorescence displacement with the JHDL compounds. We measured the relative fluorescence intensity of AGP-bound ANS in the presence of the different agents, normalizing to intensity in the absence of competing drug. When IL-3-independent growth inhibition (defined as 1 minus the ratio of growth in the absence and presence of IL-3) is compared to ANS displacement (FIG. 7B), several classes of agents are identified. Again, the majority of agents (n=2343) have neither FLT3-specific growth inhibitory nor AGP-binding activities, while a small subset (n=88) bind to AGP but are unable to restore anti-FLT3 activity. Interestingly, a small group of agents (n=91, upper right quadrant) do not appear to bind to AGP (at least not competitively with ANS), but nonetheless induce cytotoxicity that is IL-3-reversible. It is possible that these agents have intrinsic anti-FLT3 activity, or that they are synergistic with midostaurin such that the low levels of FLT3 inhibition present are now sufficient to induce cytotoxicity by either targeting alternative pathways or directly targeting downstream signaling pathways. In addition, a group of 38 agents (indicated by the dashed ellipse, upper left quadrant) are able to both bind to AGP and restore anti-FLT3 activity. There are six-fold more agents in this fourth group than would be expected based upon the number of agents found in the other groups (38 identified vs. 6.3 expected, P=2.73×10^{-23,}, Fisher's exact test). An initial validation of the 10 lead compounds demonstrated at least one, candidate 8A8, that is able to improve midostaurin activity in the presence of inhibiting AGP concentrations (FIG. 7C) to an extent similar to the effects seen with mifepristone (FIG. 6B). Testing of other candidates is in progress.

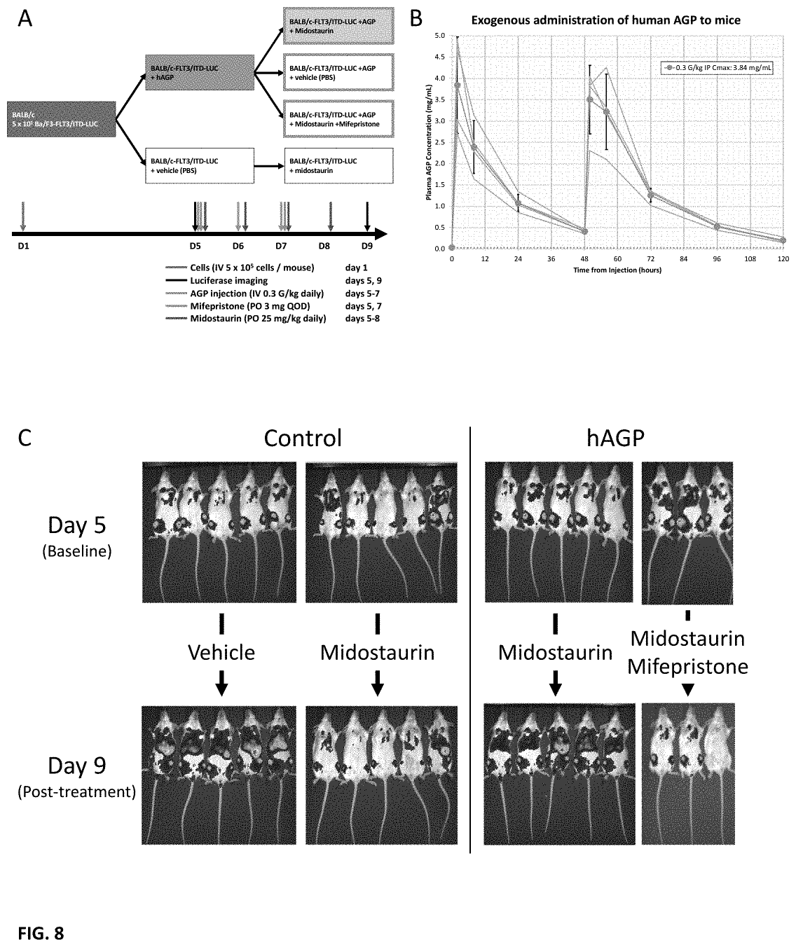

In order to test whether these disinhibiting compounds would also be effective in mice, the inventors performed an in vivo proof-of-principle of competitive disinhibition of TKI (FIG. 8A). As murine AGP has very limited homology to human AGP, is expressed at levels five- to ten-fold lower than humans, and prior work in rats has demonstrated physiologically significant differences between human and rodent AGP, we created a mouse model for testing the effects of human AGP in vivo. When mice are injected with human AGP at 0.3 g/kg intraperitoneally every 48 hours, they demonstrate serum AGP levels ranging from 0.5-3 mg/mL (FIG. 8B), with a terminal half-life of approximately 17 hours. Consistent with previous work in rats, mice do not demonstrate any appreciable toxicities from this administration over the two weeks they were observed. We transplanted sublethally-irradiated BALB/c mice with Ba/F3-FLT3/ITD-Luc cells (Ba/F3 cells expressing both FLT3/ITD and a luciferase reporter). After 5 days, adequate engraftment was confirmed by luciferin bioluminescence (FIG. 8C), and the mice were then treated with either orally gavaged midostaurin or vehicle for four days. At the same time, a subset of the midostaurin mice were also conditioned with intraperitoneal human AGP, or AGP plus mifepristone, and the remaining mice were injected with PBS vehicle. Twenty-four hours after the final treatment (day 9), the mice were again imaged to assess response (FIG. 8C). All mock-treated mice demonstrated progression of the FLT3/ITD-expressing cells whereas the midostaurin alone treated mice showed regression of the cells over the treatment course. However, treated mice that also received intraperitoneal AGP demonstrate progression of the cells comparable to that seen in the control animals. In contrast, when the midostaurin/AGP dually treated mice were also gavaged mifepristone daily during treatment, the mice again demonstrate regression of the transplanted cells at levels comparable to those of the midostaurin alone treated mice.

Since the discovery of FLT3 and its associated activating mutations, particularly FLT3/ITD, it has remained a consistent target for drug development. Two approaches, rational drug design and library-based screening, have been used for the discovery of new anti-FLT3 agents, with subsequent generations demonstrating increasing potency in vitro. These agents either tend to be staurosporine derivatives such as midostaurin and lestaurtinib or are complex poly-aromatic compounds such as gilteritinib, quizartinib and sorafenib. As a group, the staurosporines tend to be very potent, possibly due to additional off-target inhibition of targets such as PKC, and are also active against the FLT3 kinase domain mutations that have been associated with resistance to some of the FLT3 TKI that have no activity against the kinase domain mutations. However, they sometimes have significant dose-limiting toxicities, especially gastrointestinal, likely due to the same off-target effects. By contrast, some non-staurosporine agents, with more selective anti-FLT3 activity, often demonstrate higher in vitro IC₅₀s (i.e. reduced potency) and can be inhibited by more resistance-associated point mutations, but tend to be less dose-limited. Yet, despite this wide selection of diverse inhibitors spanning two decades, clinical trials have often fallen short of the expectations of pre-clinical work. In addition to prior concerns for resistance mutations, we believe that the results presented here highlight an underappreciated pitfall in the development of drugs for FLT3 AML that is likely contributing to their clinical underperformance.

One particularly informative FLT3 TKI clinical trial was that of lestaurtinib. In that randomized trial of relapsed FLT3 mutant AML patients, the addition of lestaurtinib to standard induction therapy failed to demonstrate overall improvement in response rates or overall survival. This was despite potency in vitro against FLT3/ITD cells, and patients achieving plasma drug levels nearly 1000-fold higher than necessary to demonstrate in vitro cytotoxicity. When plasma from patients on trial were directly tested for the ability to inhibit FLT3 using the original PIA, most samples failed to demonstrate high levels (>90%) of inhibitory activity. However, for those patients whose samples did achieve high levels of FLT3 inhibitory activity, there was an increase in attaining a complete remission. FLT3-inhibitory activity did not always correlate with plasma drug levels. The dissociation of drug activity from drug levels (pharmacodynamics from pharmacokinetics) was the earliest suggestion that FLT3 TKI therapy is greatly modulated by drug availability within the plasma compartment. The results presented here provide a mechanistic explanation whereby the predicted IC50 is significantly higher than most of the levels seen in patients despite excellent total drug levels.

Concerns for the effect of host factors on FLT3 TKI therapy have also been raised in response to the recent trial that led to the FDA approval of midostaurin (Rydapt). In the trial, midostaurin added to standard induction therapy yielded a modest, but significant improvement in overall survival for patients regardless of FLT3 mutational type or burden. These results are surprising in that these different FLT3 mutant disease subtypes have been demonstrated to be clinically and biologically very distinct entities, not equally dependent upon FLT3. Again, the data presented here indicate that midostaurin, too, would have appreciable anti-FLT3 activity only at concentrations significantly higher than drug levels achieved in its clinical trials. These data indicate that while midostaurin may have general anti-leukemic activity, its potent in vitro anti-FLT3 activity is largely lost in vivo, and its improvement of survival is likely through non-FLT3 targets. This is consistent with the fact that midostaurin has been well described to affect a multitude of other kinases in the kinome.

The drug-binding effects demonstrated in this report are not limited to AML. At least two other trials have experienced significant complications due to the drug-binding effects of AGP specifically. Trials with 7-hydroxy-staurosporine (UCN-01) for refractory neoplasms demonstrated disappointing results despite promising pre-clinical results. Extensive pharmacologic work identified drug binding to AGP as having been the complicating factor that was not predicted in pre-clinical models. A separate trial using the non-staurosporine TKI, filanesib (ARRY-520) for relapsed and refractory multiple myeloma met with similar results. In the filanesib trial, a very clear association between plasma AGP concentration and response was demonstrated, with the few responders to therapy having the lowest AGP concentrations of the cohort. Further complicating the clinical picture is the fact that natural AGP variants can have even greater effects upon drug activity.

These effects are not new; FDA approval involves assaying protein binding, and the literature has many similar “re-discoveries” of the problem of plasma protein binding. As drug development becomes more specialized, the health and economic consequences of ignoring drug binding effects during the pre-clinical stage of drug development and waiting until they are observed in the clinical setting can be significant. The work presented here, as well as results seen in other, unrelated models, indicate that significant differences in homology and expression levels of plasma proteins between species may mask these effects in both tissue culture and animal models. Even mouse models designed to specifically study binding to AGP utilize non-human constructs and would thus fail to predict the extent of binding and subsequent failure of the drugs in clinical trials. The inventors have shown that bovine AGP, even when adjusted to levels comparable to human plasma, produces an approximate five-fold increase in the IC50 of lestaurtinib, the most tightly bound of the staurosporines, compared to the 500-fold shift seen using human AGP. This mirrors prior work that demonstrated a protective effect of human AGP in reperfusion injury that is not found with mouse AGP. Furthermore, both cow and mouse plasma contain significantly lower concentrations (2-10 fold lower) of AGP compared with human plasma. Collectively, these results indicate the need for a new model for studying drug-protein binding that faithfully recapitulates the human effect. To our knowledge, this work is the first time such a model has been demonstrated for drug testing, and represents an exciting new platform for drug development. In the meantime, it is critical for investigators and drug developers to be aware of the limitations of current models and implement testing such as the modified PIA to avoid these failures.

The use of a second drug to increase the free-drug concentrations of the target drug to levels that are able to more fully inhibit the intended target is one way to potentially increase efficacy. An additional benefit is that by indirectly increasing drug potency, it may be possible to reduce the drug dose. This reduction in dose may lead to reduced gastrointestinal exposure and potentially reduce one of the most significant dose-limiting toxicities observed in clinical trials. Thus, ironically it may be possible to decrease drug dose while nonetheless improving efficacy. Although testing and validation continues, our screen has already demonstrated that other agents exist with properties similar to mifepristone, and agents thus identified could either form the basis of future combinatorial clinical trials, or serve as lead compounds to new, biologically inactive, plasma protein “decoys”. Indeed, even if none of these agents prove to be feasible for clinical use, this new method of screening agents and probing the drug-protein interaction space should provide a wealth of information regarding drug structure-protein interaction data that can inform both the development of new decoys as well as inform future drug development as to chemical motifs that might best be avoided during drug development. It is encouraging that mifepristone, a drug that is highly specific for the estrogen receptor, with very few off-target effects, has already shown promise both in vitro and in vivo. This suggests that this FDA-approved drug may be a candidate itself for use in a phase I trial to develop this combinatorial approach to overcoming plasma protein binding. Through better awareness of drug-protein interactions, newer models for studying the effects, and the use of combinatorial therapeutic approaches, it may be possible to finally realize the potential of targeted therapies, for FLT3 mutant AML, and for other diseases where molecularly target therapies have failed to achieve their full promise.

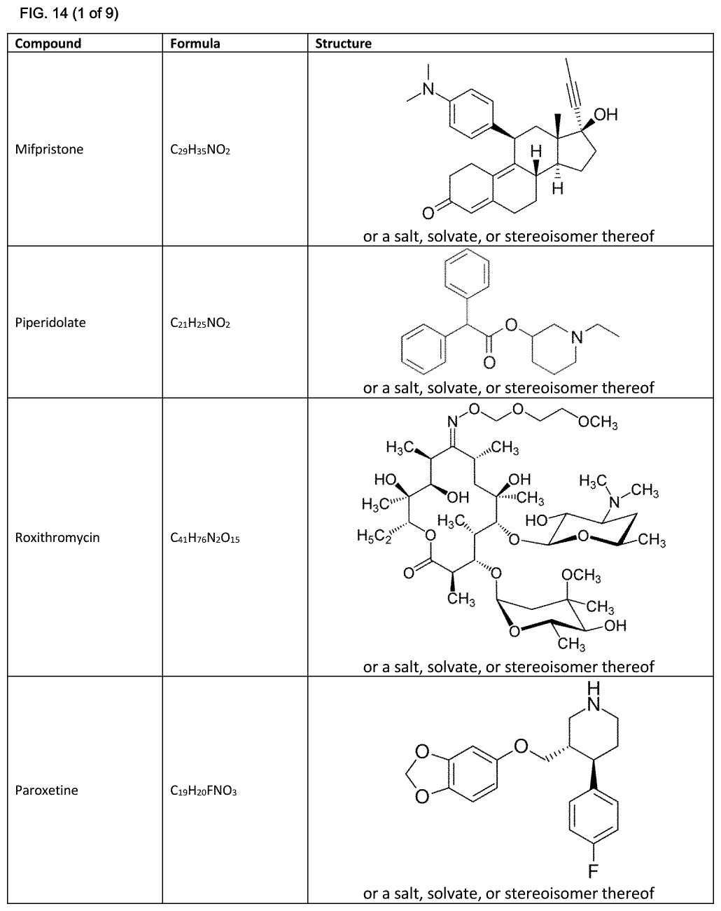

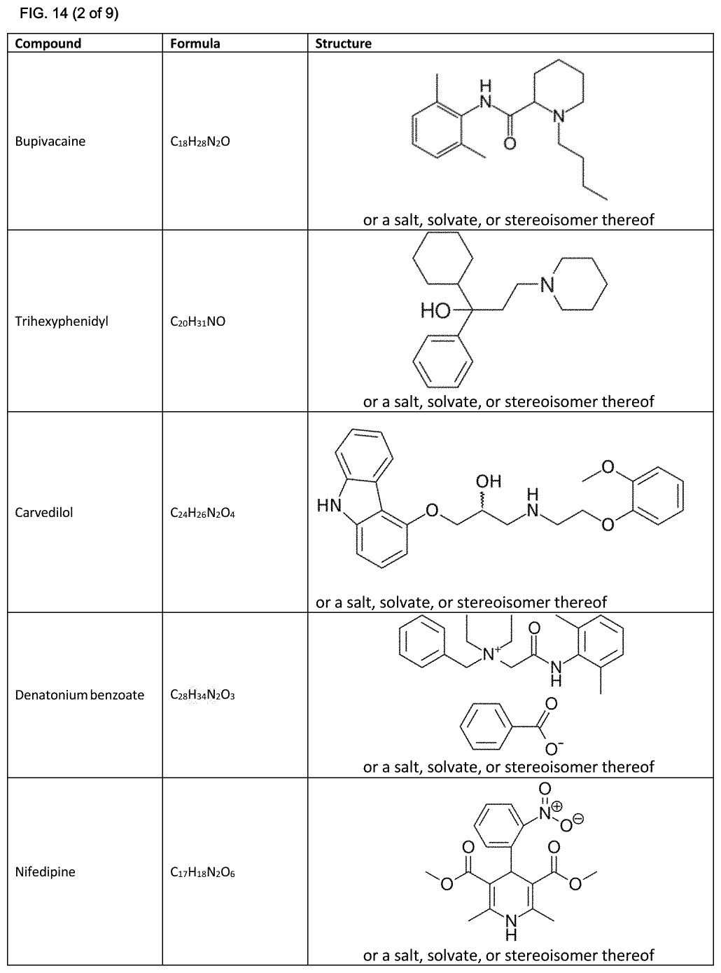

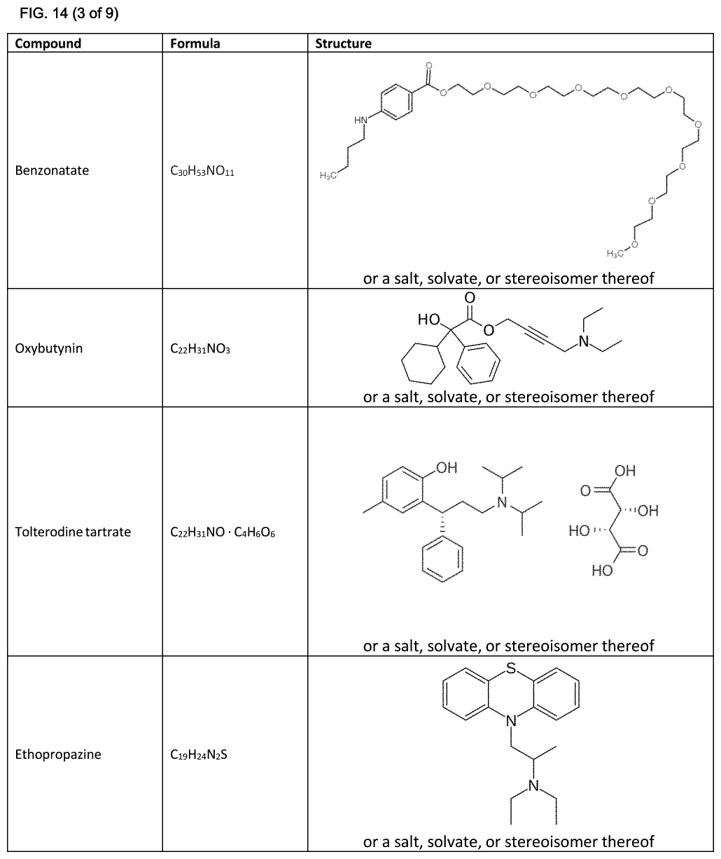

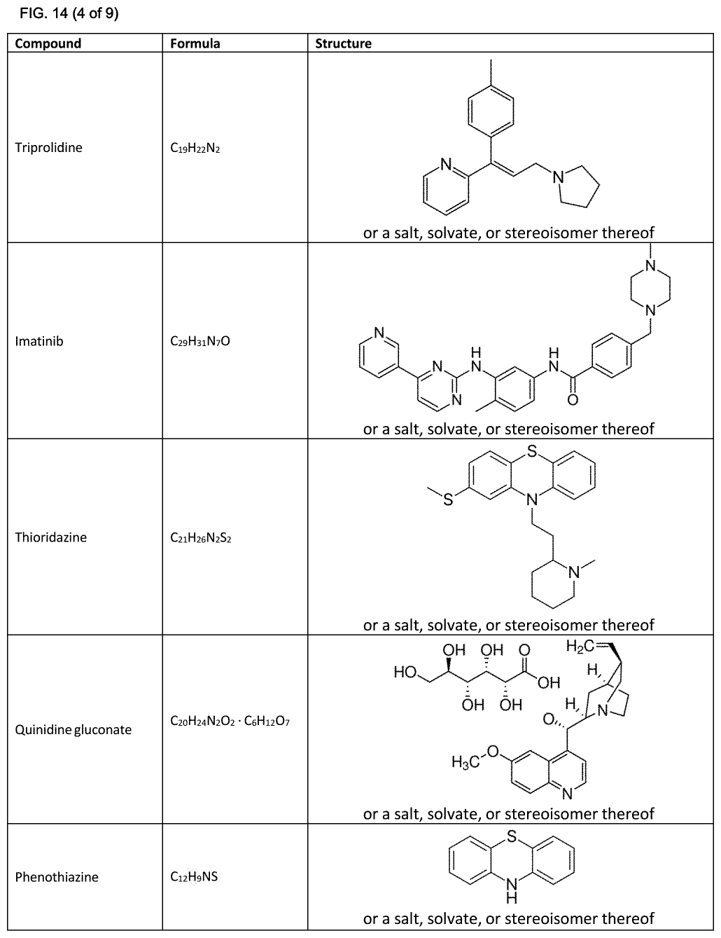

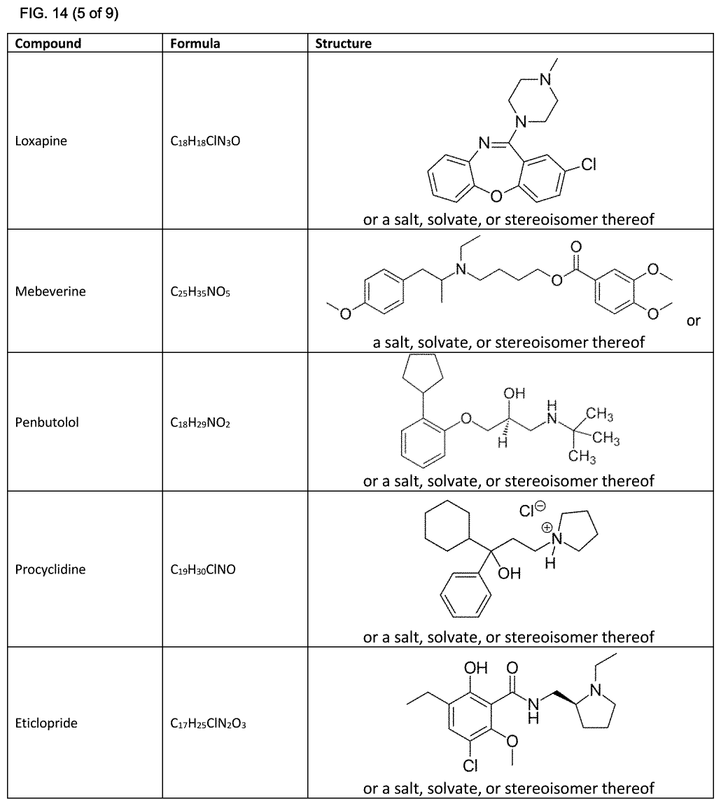

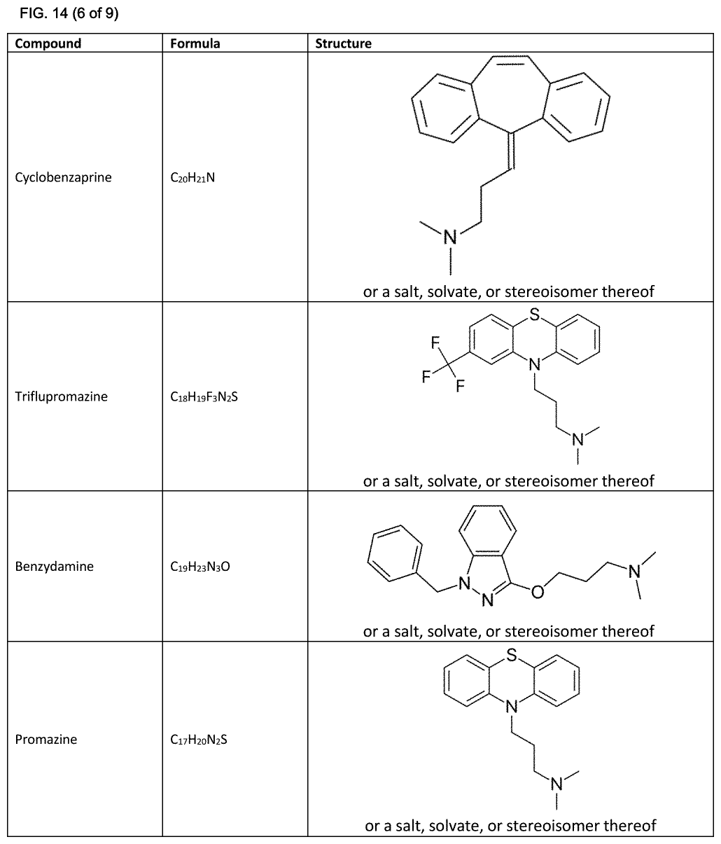

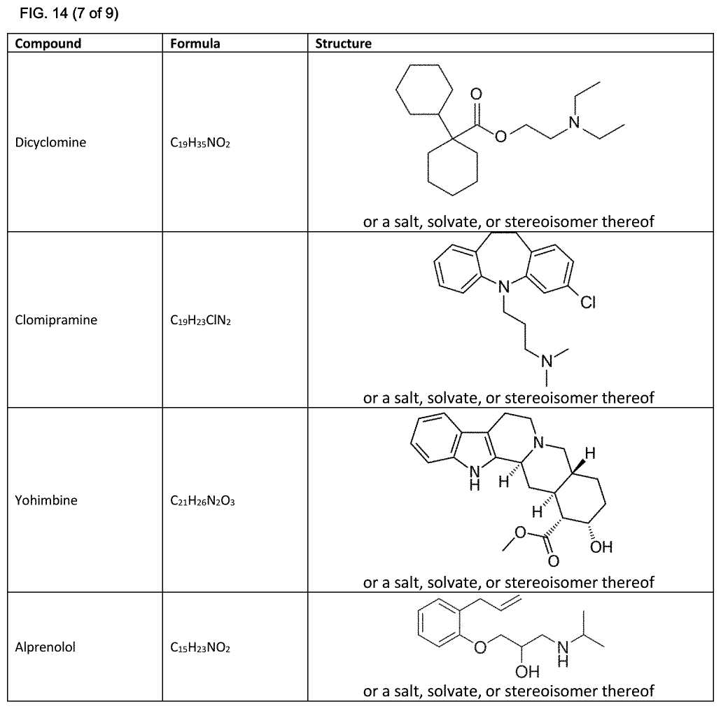

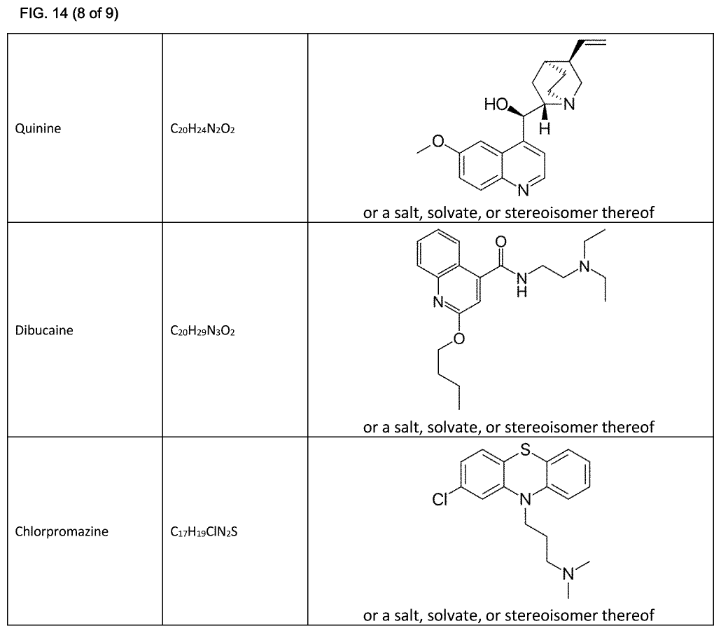

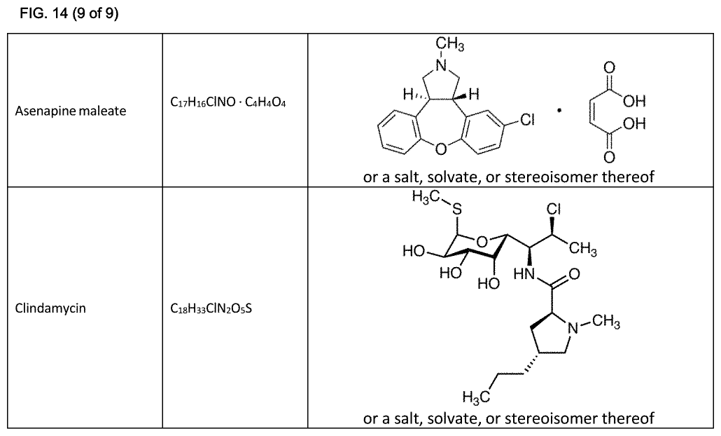

Embodiments of the disclosure concern methods and/or compositions for preventing drugs from binding to plasma proteins or releasing drugs from binding to plasma proteins, such as tyrosine kinase inhibitors, so they are able to bind to their targets. The methods of the present invention include administering to a subject a plasma protein binding agent selected from FIG. 14, or disclosed in the specification, prior to, concurrently with, or after the administration of one or more pharmaceutical agent, which binds to a plasma protein, such as tyrosine kinase inhibitor (TKI), as an example.

An individual known to be administered a drug that binds to a plasma binding protein, or will be administered a drug that binds to a plasma binding protein may be provided an effective amount of a plasma protein binding agent, such as mifepristone, for example. In particular embodiments of the disclosure, an individual is given an a pharmaceutical agent for the treatment of disease but the pharmaceutical agent binds to a plasma binding protein interfering with its ability to bind with its target. The pharmaceutical agent is administered in addition to the one or more plasma protein binding agents of the present including those listed in FIG. 14. Such additional therapy may include L-DOPA or dopamine receptor agonists and/or deep brain stimulation, for example. When combination therapy is employed, additional therapy may be given prior to, at the same time as, and/or subsequent to the plasma protein binding agent.

### Pharmaceutical Preparations

Pharmaceutical compositions of the present invention comprise an effective amount of one or more plasma protein binding agent, such as mifepristone, dissolved or dispersed in a pharmaceutically acceptable carrier. The phrases “pharmaceutical or pharmacologically acceptable” refers to molecular entities and compositions that do not produce an adverse, allergic or other untoward reaction when administered to an animal, such as, for example, a human, as appropriate. The preparation of a pharmaceutical composition that comprises at least one plasma protein binding agent or additional active ingredient will be known to those of skill in the art in light of the present disclosure, as exemplified by Remington: The Science and Practice of Pharmacy, 21st Ed. Lippincott Williams and Wilkins, 2005, incorporated herein by reference. Moreover, for animal (e.g., human) administration, it will be understood that preparations should meet sterility, pyrogenicity, general safety and purity standards as required by FDA Office of Biological Standards.

As used herein, “pharmaceutically acceptable carrier” includes any and all solvents, dispersion media, coatings, surfactants, antioxidants, preservatives (e.g., antibacterial agents, antifungal agents), isotonic agents, absorption delaying agents, salts, preservatives, drugs, drug stabilizers, gels, binders, excipients, disintegration agents, lubricants, sweetening agents, flavoring agents, dyes, such like materials and combinations thereof, as would be known to one of ordinary skill in the art (see, for example, Remington's Pharmaceutical Sciences, 18th Ed. Mack Printing Company, 1990, pp. 1289-1329, incorporated herein by reference). Except insofar as any conventional carrier is incompatible with the active ingredient, its use in the pharmaceutical compositions is contemplated.

A plasma protein binding agent may comprise different types of carriers depending on whether it is to be administered in solid, liquid or aerosol form, and whether it need to be sterile for such routes of administration as injection. The present compositions can be administered intravenously, intradermally, transdermally, intrathecally, intraarterially, intraperitoneally, intranasally, intravaginally, intrarectally, topically, intramuscularly, subcutaneously, mucosally, orally, topically, locally, inhalation (e.g., aerosol inhalation), injection, infusion, continuous infusion, localized perfusion bathing target cells directly, via a catheter, via a lavage, in cremes, in lipid compositions (e.g., liposomes), or by other method or any combination of the forgoing as would be known to one of ordinary skill in the art (see, for example, Remington's Pharmaceutical Sciences, 18th Ed. Mack Printing Company, 1990, incorporated herein by reference).

The plasma protein binding agent may be formulated into a composition in a free base, neutral or salt form. Pharmaceutically acceptable salts, include the acid addition salts, e.g., those formed with the free amino groups of a proteinaceous composition, or which are formed with inorganic acids such as for example, hydrochloric or phosphoric acids, or such organic acids as acetic, oxalic, tartaric or mandelic acid. Salts formed with the free carboxyl groups can also be derived from inorganic bases such as for example, sodium, potassium, ammonium, calcium or ferric hydroxides; or such organic bases as isopropylamine, trimethylamine, histidine or procaine. Upon formulation, solutions will be administered in a manner compatible with the dosage formulation and in such amount as is therapeutically effective. The formulations are easily administered in a variety of dosage forms such as formulated for parenteral administrations such as injectable solutions, or aerosols for delivery to the lungs, or formulated for alimentary administrations such as drug release capsules and the like.

Further in accordance with the present disclosure, the composition of the present invention suitable for administration is provided in a pharmaceutically acceptable carrier with or without an inert diluent. The carrier should be assimilable and includes liquid, semi-solid, i.e., pastes, or solid carriers. Except insofar as any conventional media, agent, diluent or carrier is detrimental to the recipient or to the therapeutic effectiveness of a the composition contained therein, its use in administrable composition for use in practicing the methods of the present invention is appropriate. Examples of carriers or diluents include fats, oils, water, saline solutions, lipids, liposomes, resins, binders, fillers and the like, or combinations thereof. The composition may also comprise various antioxidants to retard oxidation of one or more component. Additionally, the prevention of the action of microorganisms can be brought about by preservatives such as various antibacterial and antifungal agents, including but not limited to parabens (e.g., methylparabens, propylparabens), chlorobutanol, phenol, sorbic acid, thimerosal or combinations thereof.

In accordance with the present invention, the composition is combined with the carrier in any convenient and practical manner, i.e., by solution, suspension, emulsification, admixture, encapsulation, absorption and the like. Such procedures are routine for those skilled in the art. In a specific embodiment of the present invention, the composition is combined or mixed thoroughly with a semi-solid or solid carrier. The mixing can be carried out in any convenient manner such as grinding. Stabilizing agents can be also added in the mixing process in order to protect the composition from loss of therapeutic activity, i.e., denaturation in the stomach. Examples of stabilizers for use in an the composition include buffers, amino acids such as glycine and lysine, carbohydrates such as dextrose, mannose, galactose, fructose, lactose, sucrose, maltose, sorbitol, mannitol, etc.

In further embodiments, the present invention may concern the use of a pharmaceutical lipid vehicle compositions that include a plasma protein binding agent, one or more lipids, and an aqueous solvent. As used herein, the term “lipid” will be defined to include any of a broad range of substances that is characteristically insoluble in water and extractable with an organic solvent. This broad class of compounds are well known to those of skill in the art, and as the term “lipid” is used herein, it is not limited to any particular structure. Examples include compounds which contain long-chain aliphatic hydrocarbons and their derivatives. A lipid may be naturally occurring or synthetic (i.e., designed or produced by man). However, a lipid is usually a biological substance. Biological lipids are well known in the art, and include for example, neutral fats, phospholipids, phosphoglycerides, steroids, terpenes, lysolipids, glycosphingolipids, glycolipids, sulphatides, lipids with ether and ester-linked fatty acids and polymerizable lipids, and combinations thereof. Of course, compounds other than those specifically described herein that are understood by one of skill in the art as lipids are also encompassed by the compositions and methods of the present invention.

One of ordinary skill in the art would be familiar with the range of techniques that can be employed for dispersing a composition in a lipid vehicle. For example, the plasma protein binding agent may be dispersed in a solution containing a lipid, dissolved with a lipid, emulsified with a lipid, mixed with a lipid, combined with a lipid, covalently bonded to a lipid, contained as a suspension in a lipid, contained or complexed with a micelle or liposome, or otherwise associated with a lipid or lipid structure by any means known to those of ordinary skill in the art. The dispersion may or may not result in the formation of liposomes.

The actual dosage amount of a composition of the present invention administered to an animal patient can be determined by physical and physiological factors such as body weight, severity of condition, the type of disease being treated, previous or concurrent therapeutic interventions, idiopathy of the patient and on the route of administration. Depending upon the dosage and the route of administration, the number of administrations of a preferred dosage and/or an effective amount may vary according to the response of the subject. The practitioner responsible for administration will, in any event, determine the concentration of active ingredient(s) in a composition and appropriate dose(s) for the individual subject.

In certain embodiments, pharmaceutical compositions may comprise, for example, at least about 0.1% of an active compound. In other embodiments, the an active compound may comprise between about 2% to about 75% of the weight of the unit, or between about 25% to about 60%, for example, and any range derivable therein. Naturally, the amount of active compound(s) in each therapeutically useful composition may be prepared is such a way that a suitable dosage will be obtained in any given unit dose of the compound. Factors such as solubility, bioavailability, biological half-life, route of administration, product shelf life, as well as other pharmacological considerations will be contemplated by one skilled in the art of preparing such pharmaceutical formulations, and as such, a variety of dosages and treatment regimens may be desirable. In other non-limiting examples, a dose may also comprise from about 1 microgram/kg/body weight, about 5 microgram/kg/body weight, about 10 microgram/kg/body weight, about 50 microgram/kg/body weight, about 100 microgram/kg/body weight, about 200 microgram/kg/body weight, about 350 microgram/kg/body weight, about 500 microgram/kg/body weight, about 1 milligram/kg/body weight, about 5 milligram/kg/body weight, about 10 milligram/kg/body weight, about 50 milligram/kg/body weight, about 100 milligram/kg/body weight, about 200 milligram/kg/body weight, about 350 milligram/kg/body weight, about 500 milligram/kg/body weight, to about 1000 mg/kg/body weight or more per administration, and any range derivable therein. In non-limiting examples of a derivable range from the numbers listed herein, a range of about 5 mg/kg/body weight to about 100 mg/kg/body weight, about 5 microgram/kg/body weight to about 500 milligram/kg/body weight, etc., can be administered, based on the numbers described above.

### Alimentary Compositions and Formulations

In one embodiment of the present disclosure, the plasma protein binding agents are formulated to be administered via an alimentary route. Alimentary routes include all possible routes of administration in which the composition is in direct contact with the alimentary tract. Specifically, the pharmaceutical compositions disclosed herein may be administered orally, buccally, rectally, or sublingually. As such, these compositions may be formulated with an inert diluent or with an assimilable edible carrier, or they may be enclosed in hard- or soft-shell gelatin capsule, or they may be compressed into tablets, or they may be incorporated directly with the food of the diet.

In certain embodiments, the active compounds may be incorporated with excipients and used in the form of ingestible tablets, buccal tables, troches, capsules, elixirs, suspensions, syrups, wafers, and the like (Mathiowitz et al., 1997; Hwang et al., 1998; U.S. Pat. Nos. 5,641,515; 5,580,579 and 5,792, 451, each specifically incorporated herein by reference in its entirety). The tablets, troches, pills, capsules and the like may also contain the following: a binder, such as, for example, gum tragacanth, acacia, cornstarch, gelatin or combinations thereof; an excipient, such as, for example, dicalcium phosphate, mannitol, lactose, starch, magnesium stearate, sodium saccharine, cellulose, magnesium carbonate or combinations thereof; a disintegrating agent, such as, for example, corn starch, potato starch, alginic acid or combinations thereof; a lubricant, such as, for example, magnesium stearate; a sweetening agent, such as, for example, sucrose, lactose, saccharin or combinations thereof; a flavoring agent, such as, for example peppermint, oil of wintergreen, cherry flavoring, orange flavoring, etc. When the dosage unit form is a capsule, it may contain, in addition to materials of the above type, a liquid carrier. Various other materials may be present as coatings or to otherwise modify the physical form of the dosage unit. For instance, tablets, pills, or capsules may be coated with shellac, sugar, or both. When the dosage form is a capsule, it may contain, in addition to materials of the above type, carriers such as a liquid carrier. Gelatin capsules, tablets, or pills may be enterically coated. Enteric coatings prevent denaturation of the composition in the stomach or upper bowel where the pH is acidic. See, e.g., U.S. Pat. No. 5,629,001. Upon reaching the small intestines, the basic pH therein dissolves the coating and permits the composition to be released and absorbed by specialized cells, e.g., epithelial enterocytes and Peyer's patch M cells. A syrup of elixir may contain the active compound sucrose as a sweetening agent methyl and propylparabens as preservatives, a dye and flavoring, such as cherry or orange flavor. Of course, any material used in preparing any dosage unit form should be pharmaceutically pure and substantially non-toxic in the amounts employed. In addition, the active compounds may be incorporated into sustained-release preparation and formulations.

For oral administration the compositions of the present disclosure may alternatively be incorporated with one or more excipients in the form of a mouthwash, dentifrice, buccal tablet, oral spray, or sublingual orally-administered formulation. For example, a mouthwash may be prepared incorporating the active ingredient in the required amount in an appropriate solvent, such as a sodium borate solution (Dobell's Solution). Alternatively, the active ingredient may be incorporated into an oral solution such as one containing sodium borate, glycerin and potassium bicarbonate, or dispersed in a dentifrice, or added in a therapeutically-effective amount to a composition that may include water, binders, abrasives, flavoring agents, foaming agents, and humectants. Alternatively, the compositions may be fashioned into a tablet or solution form that may be placed under the tongue or otherwise dissolved in the mouth.

Additional formulations which are suitable for other modes of alimentary administration include suppositories. Suppositories are solid dosage forms of various weights and shapes, usually medicated, for insertion into the rectum. After insertion, suppositories soften, melt or dissolve in the cavity fluids. In general, for suppositories, traditional carriers may include, for example, polyalkylene glycols, triglycerides or combinations thereof. In certain embodiments, suppositories may be formed from mixtures containing, for example, the active ingredient in the range of about 0.5% to about 10%, and preferably about 1% to about 2%.

### Parenteral Compositions and Formulations

In further embodiments, plasma protein binding agents may be administered via a parenteral route. As used herein, the term “parenteral” includes routes that bypass the alimentary tract. Specifically, the pharmaceutical compositions disclosed herein may be administered for example, but not limited to intravenously, intradermally, intramuscularly, intraarterially, intrathecally, subcutaneous, or intraperitoneally U.S. Pat. Nos. 6,7537,514, 6,613,308, 5,466,468, 5,543,158; 5,641,515; and 5,399,363 (each specifically incorporated herein by reference in its entirety).

Solutions of the active compounds as free base or pharmacologically acceptable salts may be prepared in water suitably mixed with a surfactant, such as hydroxypropylcellulose. Dispersions may also be prepared in glycerol, liquid polyethylene glycols, and mixtures thereof and in oils. Under ordinary conditions of storage and use, these preparations contain a preservative to prevent the growth of microorganisms. The pharmaceutical forms suitable for injectable use include sterile aqueous solutions or dispersions and sterile powders for the extemporaneous preparation of sterile injectable solutions or dispersions (U.S. Pat. No. 5,466,468, specifically incorporated herein by reference in its entirety). In all cases the form must be sterile and must be fluid to the extent that easy injectability exists. It must be stable under the conditions of manufacture and storage and must be preserved against the contaminating action of microorganisms, such as bacteria and fungi. The carrier can be a solvent or dispersion medium containing, for example, water, ethanol, polyol (i.e., glycerol, propylene glycol, and liquid polyethylene glycol, and the like), suitable mixtures thereof, and/or vegetable oils. Proper fluidity may be maintained, for example, by the use of a coating, such as lecithin, by the maintenance of the required particle size in the case of dispersion and by the use of surfactants. The prevention of the action of microorganisms can be brought about by various antibacterial and antifungal agents, for example, parabens, chlorobutanol, phenol, sorbic acid, thimerosal, and the like. In many cases, it will be preferable to include isotonic agents, for example, sugars or sodium chloride. Prolonged absorption of the injectable compositions can be brought about by the use in the compositions of agents delaying absorption, for example, aluminum monostearate and gelatin.

For parenteral administration in an aqueous solution, for example, the solution should be suitably buffered if necessary and the liquid diluent first rendered isotonic with sufficient saline or glucose. These particular aqueous solutions are especially suitable for intravenous, intramuscular, subcutaneous, and intraperitoneal administration. In this connection, sterile aqueous media that can be employed will be known to those of skill in the art in light of the present disclosure. For example, one dosage may be dissolved in isotonic NaCl solution and either added hypodermoclysis fluid or injected at the proposed site of infusion, (see for example, “Remington's Pharmaceutical Sciences” 15th Edition, pages 1035-1038 and 1570-1580). Some variation in dosage will necessarily occur depending on the condition of the subject being treated. The person responsible for administration will, in any event, determine the appropriate dose for the individual subject. Moreover, for human administration, preparations should meet sterility, pyrogenicity, general safety and purity standards as required by FDA Office of Biologics standards.

Sterile injectable solutions are prepared by incorporating the active compounds in the required amount in the appropriate solvent with various of the other ingredients enumerated above, as required, followed by filtered sterilization. Generally, dispersions are prepared by incorporating the various sterilized active ingredients into a sterile vehicle which contains the basic dispersion medium and the required other ingredients from those enumerated above. In the case of sterile powders for the preparation of sterile injectable solutions, the preferred methods of preparation are vacuum-drying and freeze-drying techniques which yield a powder of the active ingredient plus any additional desired ingredient from a previously sterile-filtered solution thereof. A powdered composition is combined with a liquid carrier such as, e.g., water or a saline solution, with or without a stabilizing agent.

### Miscellaneous Pharmaceutical Compositions and Formulations

In other preferred embodiments of the invention, the active compound, the plasma protein binding agent, may be formulated for administration via various miscellaneous routes, for example, topical (i.e., transdermal) administration, mucosal administration (intranasal, vaginal, etc.) and/or inhalation. Pharmaceutical compositions for topical administration may include the active compound formulated for a medicated application such as an ointment, paste, cream or powder. Ointments include all oleaginous, adsorption, emulsion and water-solubly based compositions for topical application, while creams and lotions are those compositions that include an emulsion base only. Topically administered medications may contain a penetration enhancer to facilitate adsorption of the active ingredients through the skin. Suitable penetration enhancers include glycerin, alcohols, alkyl methyl sulfoxides, pyrrolidones and luarocapram. Possible bases for compositions for topical application include polyethylene glycol, lanolin, cold cream and petrolatum as well as any other suitable absorption, emulsion or water-soluble ointment base. Topical preparations may also include emulsifiers, gelling agents, and antimicrobial preservatives as necessary to preserve the active ingredient and provide for a homogenous mixture. Transdermal administration of the present invention may also comprise the use of a “patch”. For example, the patch may supply one or more active substances at a predetermined rate and in a continuous manner over a fixed period of time.

In certain embodiments, the pharmaceutical compositions may be delivered by eye drops, intranasal sprays, inhalation, and/or other aerosol delivery vehicles. Methods for delivering compositions directly to the lungs via nasal aerosol sprays has been described e.g., in U.S. Pat. Nos. 5,756,353 and 5,804,212 (each specifically incorporated herein by reference in its entirety). Likewise, the delivery of drugs using intranasal microparticle resins (Takenaga et al., 1998) and lysophosphatidyl-glycerol compounds (U.S. Pat. No. 5,725, 871, specifically incorporated herein by reference in its entirety) are also well-known in the pharmaceutical arts. Likewise, transmucosal drug delivery in the form of a polytetrafluoroetheylene support matrix is described in U.S. Pat. No. 5,780,045 (specifically incorporated herein by reference in its entirety). The term aerosol refers to a colloidal system of finely divided solid of liquid particles dispersed in a liquefied or pressurized gas propellant. The typical aerosol of the present invention for inhalation will consist of a suspension of active ingredients in liquid propellant or a mixture of liquid propellant and a suitable solvent. Suitable propellants include hydrocarbons and hydrocarbon ethers. Suitable containers will vary according to the pressure requirements of the propellant. Administration of the aerosol will vary according to subject's age, weight and the severity and response of the symptoms.

### EXAMPLES/METHODS

The following Examples/Methods have been included to provide guidance to one of ordinary skill in the art for practicing representative embodiments of the presently disclosed subject matter. In light of the present disclosure and the general level of skill in the art, those of skill can appreciate that the following Examples/Methods are intended to be exemplary only and that numerous changes, modifications, and alterations can be employed without departing from the scope of the presently disclosed subject matter. The following Examples are offered by way of illustration and not by way of limitation.

### Patient Plasma

Samples were collected from patients and healthy donors seen in the pediatric oncology clinic at Johns Hopkins Hospital between November 2015 and March 2016. Patients were enrolled in an IRB-approved, institutional banking protocol with informed patient consent in accordance with the Declaration of Helsinki. Whole blood is collected by venipuncture in sodium heparin-treated vacutainer tubes (BD) and then processed within 2 hours of collection. Cellular components are separated from the plasma by centrifugation twice at 2,000×g for 10 minutes. Samples are stored until use at −80° C., and clarified immediately prior to use by centrifugation at ≥15,000×g for 3 minutes. Plasma alpha-1-acid glycoprotein concentrations were measured by radial immunodiffusion assay (Kent Labs) according to the manufacturer's protocols.

### Reagents

Alpha-1-acid glycoprotein (AGP) purified from human or bovine plasma (Sigma) was resuspended in unsupplemented RPMI 1640 (Gibco) at 10 mg/mL for working stocks. Recombinant human serum albumin (Sigma) was resuspended in RPMI 1640 at 100 mg/mL. Lyophilized bovine plasma (Sigma) was reconstituted in sterile PBS (Gibco). TTT-3002 was a generous gift of TauTaTis, Inc. (San Diego, Calif.). Lestaurtinib, midostaurin, sorafenib and quizartinib were purchased from LC Laboratories. Each FLT3 TKI was dissolved at 10 mM in 100% sterile-filtered dimethylsulfoxide (DMSO, Sigma). Mifepristone (Sigma) was dissolved at 100 mM in 100% DMSO. Trihexyphenidyl hydrochloride (Selleckchem) was dissolved at 100 mM in methanol. For cell-based assays, working stocks of 10 μM were prepared for each drug in RPMI 1640 supplemented with 0.1% DMSO and 0.2% bovine serum albumin. For spectrophotometric studies, working stocks of 10 μM were prepared using PBS without sera or albumin. 8-anilinonaphthalenel-sulfonic acid (ANS, Sigma) was dissolved in DMSO at 200 mM, and then diluted to 400 μM with PBS (0.2% DMSO, final).

### Cell Lines

Unless otherwise stated, MOLM-14 cells (DSMZ) and Ba/F3 (ATCC) are grown in RPMI 1640 media, supplemented with 10% heat-inactivated fetal bovine serum (FBS, Gemini), with antibiotics. The Ba/F3-FLT3/ITD cell line is a Ba/F3 cell line into which the neomycin-selectable pBABE vector with FLT3/ITD has been stably incorporated, as previously described.(31,55) Ba/F3-FLT3/ITD-Luc cell line was the BaF3 FLT3/ITD cells transfected with the L3-3GFP plasmid containing genes for luciferase and green fluorescent protein (GFP).(55) Parental Ba/F3 cells are cultured with 1 ng/mL recombinant mouse IL-3, whereas Ba/F3-FLT3/ITD and Ba/F3-FLT3/ITD-Luc cells are maintained without supplemental cytokines. Cells are maintained at 4×10⁵-2×10⁶ cells per milliliter and cultured in humidified, 37° C. incubators with 5% carbon dioxide.

### Modified Plasma Inhibition Assay

Cells in logarithmic growth are resuspended in either human plasma or serum-free RPMI 1640 media supplemented with the plasma protein of interest and seeded in 96-well format at 50 μL per well. Cell density is 2×10⁵ cells per well for MOLM-14 and Ba/F3-FLT3/ITD cells. To each well 50 μL of 2× drug dilution in RPMI 1640 media with 10% serum was added. Each condition is plated in quadruplicate. The cells are then incubated at 37° C. After 44 hours of treatment, 10 μL of 5 mg/mL thiazolyl blue tetrazolium bromide (MTT, Sigma) in PBS, sterile-filtered, is added to each well, and the plates are incubated for an additional 4 hours at 37° C. 100 of 10% sodium dodecyl sulfate (JT Baker) in 10 mM hydrochloric acid (Fisher) is then added to each well, and the plates are incubated at 37° C. overnight. Optical absorbance at 570 nm is measured via plate reader (iMark, Bio-Rad), averaging across technical replicates. 50% inhibition of proliferation (IC₅₀) values were calculated by linear regression analysis relative to cells cultured without drug(s) in the corresponding presence or absence of plasma or plasma proteins.

### Apoptosis Assays

MOLM-14 cells in logarithmic growth are resuspended in either human plasma or RPMI 1640 media supplemented with 10% FBS and the plasma protein of interest at a cell density of 5×10⁵ cells per milliliter and plated in 6-well format. Drug was added at an appropriate concentration, and the cells were incubated for 24 hours at 37° C. Cells were then collected, washed, and stained with phycoerythrin-conjugated Annexin V and 7-amino-actinomycin D (BD Biosciences) per manufacturer's protocols and analyzed by flow cytometry. A fraction of cells was also collected for Western blot analysis for FLT3 autophosphorylation and poly (ADP-ribose) polymerase (PARP) cleavage as below.

### Western Blot Analysis of Cell Signaling

Cells in logarithmic growth are resuspended in either plasma or RPMI 1640 media supplemented with either 10% FBS serum or physiologic concentrations of purified plasma proteins. The cells are cultured for 1 hour at 37° C. in the presence of drug(s) with intermittent shaking. The cells are then washed, lysed with RIPA buffer (Sigma) per manufacturer's protocol, and total cell lysates separated by sodium dodecyl sulfate polyacrylamide gel electrophoresis (Invitrogen) with Western transfer onto polyvinyl difluoride membranes (Millipore). The membranes are probed for FLT3 by using the S-18 antibody (Santa Cruz), or for other proteins using the indicated antibodies (Cell Signaling Technology), followed by horseradish peroxidase-conjugated goat anti-rabbit secondary antibodies with enhanced chemiluminescence detection (Bio-Rad).

### Fluorescence Displacement Titration

Affinity of drugs for AGP is measured by displacement of ANS from AGP using the previously described fluorescent spectrophotometric method. Serial dilutions of ANS in PBS are made, and the drug of interest (dissolved in serum-free PBS) is combined with AGP and ANS dilutions for final concentrations of 1 μM drug, 0.5 mg/mL AGP and ANS at a range of 90 nM to 50 μM in a total of 200 μL in quadruplicate. The drugs and protein are allowed to equilibrate at room temperature for 2 hours. Fluorescence is then measured at 470 nm, with an excitation at 400 nm with a SpectraMax M3 Multi-Mode Microplate reader (Molecular Devices). Maximal fluorescence is determined by measuring ANS fluorescence in the presence of 100 μM human serum albumin (Sigma) without drug. Bound ANS is calculated as the product of total ANS concentration times the fraction of maximal fluorescence observed. The association constant (K_{A}) of ANS with AGP in the absence of drug is calculated by Scatchard analysis, and the binding constants of drug with AGP are subsequently determined by the Cheng-Prusoff approximation.(56)

### Animal Studies

BALB/c mice were transplanted after 300 cGy of sublethal X-ray irradiation using a CIXD irradiator (Xstrahl). 5×10⁵ Ba/F3-FLT3/ITD-Luc cells were injected via tail vein. Intraperitoneal injections were performed using PBS vehicle with human AGP (Sigma) at 20 mg/mL. Midostaurin was suspended in 30%(w/v) Cremophor EL, 30% (w/v) PEG 400, 10% ethanol, and 10% glucose (all Sigma), and instilled once daily by oral gavage (16,57). These drug doses have previously been demonstrated to be effective against FLT3/ITD in mice. Mifepristone (Sigma) was dissolved in 50% ethanol and 50% (2-hydroxypropyl)-□-cyclodextrin (Sigma) and administered by oral gavage every 48 hours. Mice were imaged by intraperitoneal injection of luciferin (3 mg) and visualizing on an IVIS Spectrum imager (Caliper LifeSciences) using Living Image software for analysis on day 5 (to monitor engraftment) and on day 9 (to assess drug effect). All animal husbandry and procedures were conducted in accordance with the policy of the Johns Hopkins University School of Medicine Animal Care and Use Committee.

All references, including publications, patent applications, and patents, cited herein are hereby incorporated by reference to the same extent as if each reference were individually and specifically indicated to be incorporated by reference and were set forth in its entirety herein.

The use of the terms “a” and “an” and “the” and similar referents in the context of describing the invention (especially in the context of the following claims) are to be construed to cover both the singular and the plural, unless otherwise indicated herein or clearly contradicted by context. The terms “comprising,” “having,” “including,” and “containing” are to be construed as open-ended terms (i.e., meaning “including, but not limited to,”) unless otherwise noted. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

Preferred embodiments of this invention are described herein, including the best mode known to the inventors for carrying out the invention. Variations of those preferred embodiments may become apparent to those of ordinary skill in the art upon reading the foregoing description. The inventors expect skilled artisans to employ such variations as appropriate, and the inventors intend for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

What is claimed is:

1. A method of inhibiting one or more first pharmaceutical agent from binding to a plasma protein comprising the steps of administering to a subject a plasma protein binding agent prior to, concurrently with, or after the administration of a one or more pharmaceutical agent that binds to a plasma protein in vivo.

2. The method of claim 1 wherein the plasma protein binding agent is Mifepristone, Piperidolate, Roxithromycin, Paroxetine, Bupivacaine, Trihexyphenidyl, Carvedilol, Denatonium, Nifedipine, Benzonatate, Oxybutynin, Tolterodine tartate, Ethopropazine, Triprolidine, Imatinib, Thioridazine, Quinidine gluconate, Phenothiazine, Loxapine, Mebeverine, Penbutol, Procyclidine, Eticlopride, Cyclobenzaprine, Triflupromazine, Benzydamine, Promazine, Dicyclomine, Clomipramine, Yohimbine, Alprenolol, Quinine, Dibucaine, Chlorpromazine, Asenapine, or Clindamycin, or a salt, solvate, or stereoisomer thereof, or a combination thereof.

3. The method of claim 1 wherein the plasma protein binding agent is Mispristone, Roxithromycin, Bupivacaine, Bupivacaine, Imatib Mesylate, Phenothiazine, or Mebeverine, or a salt, solvate, or stereoisomer thereof, or a combination thereof.

4. The method of claim 1 wherein the plasma protein binding agent is mifepristone.

5. The method of claim 1 wherein the pharmaceutical agent is a tyrosine kinase inhibitor (TKI).

6. The method of claim 5 wherein the tyrosine kinase inhibitor (TKI) is selected from the group consisting of TT-3002, lestaurtinib, midostaurin, midostaurin, and sorafenib, staurosporine-derived TKI, anti-FLT3 agents, or a salt, solvate, or stereoisomer thereof, and a combination thereof.

7. The method of claim 5 wherein the disease is acute myeloid leukemia (AML), refractory neoplasms, or multiple myeloma.

8. A method of inhibiting one or more pharmaceutical agent from binding to a plasma protein and treating a disease comprising the steps of:
administering to a subject a plasma protein binding agent prior to, concurrently with, or after administration of the one or more pharmaceutical agent to the subject to treat a disease of the subject;
binding of the plasma protein binding agent to a plasma protein so that the one or more pharmaceutical agent is unable to bind to the plasma protein; and
treating the disease of the subject.

9. The method of claim 8 wherein the disease is acute myeloid leukemia (AML), refractory neoplasms, and multiple myeloma.

10. A method of releasing one or more first pharmaceutical agent bound to a plasma protein comprising the steps of administering to a subject a plasma protein binding agent prior to, concurrently with, or after the administration of a one or more pharmaceutical agent that binds to a plasma protein in vivo.

11. The method of claim 10 wherein the plasma protein binding agent is Mifepristone, Piperidolate, Roxithromycin, Paroxetine, Bupivacaine, Trihexyphenidyl, Carvedilol, Denatonium, Nifedipine, Benzonatate, Oxybutynin, Tolterodine tartate, Ethopropazine, Triprolidine, Imatinib, Thioridazine, Quinidine gluconate, Phenothiazine, Loxapine, Mebeverine, Penbutol, Procyclidine, Eticlopride, Cyclobenzaprine, Triflupromazine, Benzydamine, Promazine, Dicyclomine, Clomipramine, Yohimbine, Alprenolol, Quinine, Dibucaine, Chlorpromazine, Asenapine, or Clindamycin, or a salt, solvate, or stereoisomer thereof, or a combination thereof.

12. The method of claim 10 wherein the plasma protein binding agent is Mispristone, Roxithromycin, Bupivacaine 4, Bupivacaine hydrochloride, Imatib Mesylate, Phenothiazine 4, Mebeverine hydrochloride,

13. The method of claim 10 wherein the plasma protein binding agent is mifepristone.

14. The method of claim 10 wherein the pharmaceutical agent is a tyrosine kinase inhibitor (TKI).

15. The method of claim 14 wherein the tyrosine kinase inhibitor (TKI) is selected from the group consisting of TT-3002, lestaurtinib, midostaurin, midostaurin, and sorafenib, staurosporine-derived TKI, anti-FLT3 agents, or a salt, solvate, or stereoisomer thereof, and a combination thereof.

16. The method of claim 14 wherein the disease is AML, refractory neoplasms, or multiple myeloma.

17. A method of releasing one or more pharmaceutical agent bound to a plasma protein and treating a disease comprising the steps of:
administering to a subject a plasma protein binding agent prior to, concurrently with, or after administration of the one or more pharmaceutical agent to the subject to treat a disease of the subject;
unbinding the one or more pharmaceutical agent bound to a plasma protein; and
treating the disease of the subject.

18. The method of claim 17 wherein the disease is acute myeloid leukemia (AML), refractory neoplasms, or multiple myeloma.

19. The method of claim 17 further comprising the step of forming an unbound pharmaceutical agent that binds to a target.
